SEQUENCE LISTING (50 sequences)

SEQ ID NO: 1 - RNA, 21 nt, synthetic construct
  source 1..21: mol_type = other RNA
aagttgaactgctagcctctg

SEQ ID NO: 2 - RNA, 21 nt, synthetic construct
  source 1..21: mol_type = other RNA
ttccgccactgaacattggaa

SEQ ID NO: 3 - RNA, 21 nt, synthetic construct
  source 1..21: mol_type = other RNA
atctataatgatcaggttcat

SEQ ID NO: 4 - RNA, 21 nt, synthetic construct
  source 1..21: mol_type = other RNA
aagttccgccactgaacattg

SEQ ID NO: 5 - RNA, 21 nt, synthetic construct
  source 1..21: mol_type = other RNA
tccgccactgaacattggaat

SEQ ID NO: 6 - RNA, 21 nt, synthetic construct
  source 1..21: mol_type = other RNA
caagttccgccactgaacatt

SEQ ID NO: 7 - RNA, 21 nt, synthetic construct
  source 1..21: mol_type = other RNA
agttccgccactgaacattgg

SEQ ID NO: 8 - RNA, 21 nt, synthetic construct
  source 1..21: mol_type = other RNA
gttccgccactgaacattgga

SEQ ID NO: 9 - RNA, 21 nt, synthetic construct
  source 1..21: mol_type = other RNA
acatagcgcctctgactggga

SEQ ID NO: 10 - RNA, 21 nt, synthetic construct
  source 1..21: mol_type = other RNA
aggttcattgtcactaacatc

SEQ ID NO: 11 - RNA, 21 nt, synthetic construct
  source 1..21: mol_type = other RNA
aggattgcaagttccgccact

SEQ ID NO: 12 - RNA, 21 nt, synthetic construct
  source 1..21: mol_type = other RNA
gaagttgaactgctagcctct

SEQ ID NO: 13 - RNA, 21 nt, synthetic construct
  source 1..21: mol_type = other RNA
caggttcattgtcactaacat

SEQ ID NO: 14 - RNA, 21 nt, synthetic construct
  source 1..21: mol_type = other RNA
atagcgcctctgactgggaat

SEQ ID NO: 15 - RNA, 21 nt, synthetic construct
  source 1..21: mol_type = other RNA
gattgcaagttccgccactga

SEQ ID NO: 16 - RNA, 21 nt, synthetic construct
  source 1..21: mol_type = other RNA
caggtagctataataatacac

SEQ ID NO: 17 - RNA, 21 nt, synthetic construct
  source 1..21: mol_type = other RNA
gctagcctctggatttgacgg

SEQ ID NO: 18 - RNA, 21 nt, synthetic construct
  source 1..21: mol_type = other RNA
agcgcctctgactgggaatag

SEQ ID NO: 19 - RNA, 21 nt, synthetic construct
  source 1..21: mol_type = other RNA
gtggtgtcagaatatctataa

SEQ ID NO: 20 - RNA, 21 nt, synthetic construct
  source 1..21: mol_type = other RNA
ctggtcctggtatgaagaatg

SEQ ID NO: 21 - RNA, 21 nt, synthetic construct
  source 1..21: mol_type = other RNA
cctttagctggcagaccacaa

SEQ ID NO: 22 - RNA, 21 nt, synthetic construct
  source 1..21: mol_type = other RNA
ccgccactgaacattggaata

SEQ ID NO: 23 - RNA, 21 nt, synthetic construct
  source 1..21: mol_type = other RNA
ggtgtcagaatatctataatg

SEQ ID NO: 24 - RNA, 19 nt, synthetic construct
  source 1..19: mol_type = other RNA
gaggctagcagttcaactt

SEQ ID NO: 25 - RNA, 19 nt, synthetic construct
  source 1..19: mol_type = other RNA
ccaatgttcagtggcggaa

SEQ ID NO: 26 - RNA, 19 nt, synthetic construct
  source 1..19: mol_type = other RNA
gaacctgatcattatagat

SEQ ID NO: 27 - RNA, 19 nt, synthetic construct
  source 1..19: mol_type = other RNA
atgttcagtggcggaactt

SEQ ID NO: 28 - RNA, 19 nt, synthetic construct
  source 1..19: mol_type = other RNA
tccaatgttcagtggcgga

SEQ ID NO: 29 - RNA, 19 nt, synthetic construct
  source 1..19: mol_type = other RNA
tgttcagtggcggaacttg

SEQ ID NO: 30 - RNA, 19 nt, synthetic construct
  source 1..19: mol_type = other RNA
aatgttcagtggcggaact

SEQ ID NO: 31 - RNA, 19 nt, synthetic construct
  source 1..19: mol_type = other RNA
caatgttcagtggcggaac

SEQ ID NO: 32 - RNA, 19 nt, synthetic construct
  source 1..19: mol_type = other RNA
ccagtcagaggcgctatgt

SEQ ID NO: 33 - RNA, 19 nt, synthetic construct
  source 1..19: mol_type = other RNA
tgttagtgacaatgaacct

SEQ ID NO: 34 - RNA, 19 nt, synthetic construct
  source 1..19: mol_type = other RNA
tggcggaacttgcaatcct

SEQ ID NO: 35 - RNA, 19 nt, synthetic construct
  source 1..19: mol_type = other RNA
aggctagcagttcaacttc

SEQ ID NO: 36 - RNA, 19 nt, synthetic construct
  source 1..19: mol_type = other RNA
gttagtgacaatgaacctg

SEQ ID NO: 37 - RNA, 19 nt, synthetic construct
  source 1..19: mol_type = other RNA
tcccagtcagaggcgctat

SEQ ID NO: 38 - RNA, 19 nt, synthetic construct
  source 1..19: mol_type = other RNA
agtggcggaacttgcaatc

SEQ ID NO: 39 - RNA, 19 nt, synthetic construct
  source 1..19: mol_type = other RNA
gtattattatagctacctg

SEQ ID NO: 40 - RNA, 19 nt, synthetic construct
  source 1..19: mol_type = other RNA
gtcaaatccagaggctagc

SEQ ID NO: 41 - RNA, 19 nt, synthetic construct
  source 1..19: mol_type = other RNA
attcccagtcagaggcgct

SEQ ID NO: 42 - RNA, 19 nt, synthetic construct
  source 1..19: mol_type = other RNA
atagatattctgacaccac

SEQ ID NO: 43 - RNA, 19 nt, synthetic construct
  source 1..19: mol_type = other RNA
ttcttcataccaggaccag

SEQ ID NO: 44 - RNA, 19 nt, synthetic construct
  source 1..19: mol_type = other RNA
gtggtctgccagctaaagg

SEQ ID NO: 45 - RNA, 19 nt, synthetic construct
  source 1..19: mol_type = other RNA
ttccaatgttcagtggcgg

SEQ ID NO: 46 - RNA, 19 nt, synthetic construct
  source 1..19: mol_type = other RNA
ttatagatattctgacacc

SEQ ID NO: 47 - RNA, 20 nt, synthetic construct
  source 1..20: mol_type = other RNA
ttctgtttgtggaagaactc

SEQ ID NO: 48 - RNA, 20 nt, synthetic construct
  source 1..20: mol_type = other RNA
gagttcttccacaaacagaa

SEQ ID NO: 49 - RNA, 20 nt, synthetic construct
  source 1..20: mol_type = other RNA
ttctgtttgtggaagaactc

SEQ ID NO: 50 - RNA, 15 nt, synthetic construct
  source 1..15: mol_type = other RNA
cttccacaaacagaa

(19) United States
(12) Patent Application Publication
(10) Pub. No.: US 2025/0136992 A1
(43) Pub. Date: May 1, 2025

(54) ANTI-PTEN RNA INTERFERENCE OLIGONUCLEOTIDES AND USES THEREOF

(71) Applicant: NUREXONE BIOLOGIC LTD., Haifa (IL)

(72) Inventors: Nisim PERETS, Tel Aviv (IL); Lyora AHARONOV, Haifa (IL); Lior SHALTIEL, Modi'in (IL); Josef MOGRABI, Tel Aviv (IL)

(21) Appl. No.: 18/865,940

(22) PCT Filed: May 14, 2023
(86) PCT No.: PCT/IL2023/050494

§ 371 Date: Nov. 14, 2024

Related U.S. Application Data
(60) Provisional application No. 63/342,112, filed on May 15, 2022.

(51) Int. Cl. C12N 15/113; A61P 25/00; C12N 15/11

(52) U.S. Cl. CPC C12N 15/1137; A61P 25/00; C12N 15/111; C12N 2310/122; C12N 2310/14; C12N 2310/3515

(57) ABSTRACT

The present invention provides RNA interference (RNAi) oligonucleotides inhibiting expression of Phosphatase and tensin homolog (PTEN), extracellular vesicles comprising the RNAi oligonucleotides, pharmaceutical compositions including the RNAi oligonucleotides or said extracellular vesicles and their use in the treatment of neurological and CNS disorders and, more particularly to their use for the treatment of spinal cord injuries.

### FIELD OF THE INVENTION

The present invention relates to RNA interference oligonucleotides inhibiting expression of PTEN, extracellular vesicles comprising same, pharmaceutical compositions including the RNAi or said vesicles and their use in the treatment of neurological disorders and, more particularly to their use for the treatment of spinal cord injuries.

### BACKGROUND OF THE INVENTION

Damage to the spinal cord may result in autonomic dysfunction, a loss of sensation or a loss of mobility. Such spinal cord injury (SCI) is caused by trauma, tumors, ischemia, developmental disorders, neurodegenerative diseases, demyelinating diseases, transverse myelitis, vascular malformations, or other causes. The consequences of SCI depend on the specific nature of the injury and its location along the spinal cord. In addition, because SCI is a dynamic process, the full extent of injury may not be apparent initially in all acute cord syndromes. Incomplete cord lesions may evolve into more complete lesions; more commonly, the injury raises one or two spinal levels during the hours to days after the initial event. A complex cascade of pathophysiologic events accounts for this clinical deterioration.

The psychological and social impact of SCIs often is devastating. Some of the general disabling conditions associated with SCI are permanent paralysis of the limbs, chronic pain, muscular atrophy, loss of voluntary control over the bladder and bowel, sexual dysfunction, and infertility.

Recent advances in neuroscience have drawn considerable attention to research into SCI and have made significantly better treatment and rehabilitation options available. Functional electrical stimulation (FES), for example, has shown the potential to enhance nerve regeneration and allow significant improvements in restoring and improving functional capacity after SCI. However, not all patients with spinal cord injury qualify for FES; a complete lesion of the spinal cord must be present; the patient must be in a neurologically stable condition; and the peripheral nerves must be intact to respond to exogenous electrical stimulations.

Axon regeneration following SCI is limited, due to intrinsic extremely limited ability of mature neurons to grow, and also extrinsic factors, such as glial scar maturing overtime and inhibitory molecules. Attempts have been made to modify extrinsic factors, yet success is limited. For example, extracellular inhibitory molecule removal, neurotrophic factor delivery, or permissive substrate grafting failed to elicit robust regeneration of injured corticospinal tract.

Phosphatase and tensin homolog (PTEN) is a highly conserved dual-specificity protein tyrosine phosphatase. The protein dephosphorylates the lipid second messengers phosphatidylinositol 3,4,5-trisphosphate [PI(3,4,5)P3] and phosphatidylinositol 3,4-bisphosphate [PI(3,4)P2] to produce phosphatidylinositol 4,5-bisphosphate [PI(4,5)P2] and phosphatidylinositol 4-phosphate [PI(4) P], respectively. This unique activity makes PTEN a major homeostatic regulator and tumor suppressor protein, which function is absent or defective in a wide variety of tumors as a result of somatic alterations. The important role of the PI3K/AKT/mTOR signaling pathway in cell growth, regeneration, and survival supports the rationale for the therapeutic targeting of PTEN. Among the suggested PTEN inhibition-based therapeutic targets are nerve growth and regeneration after injury or damage, treatment of cardiac ischemia/reperfusion and associated disease, wound repair, and infertility (see review by Pulido, 2018, Molecules, 23, 285). Interestingly, the main paradigm of PTEN involvement in cancer is as a cancer suppressor, and it has been shown that PTEN inhibition may occur in brain metastases (Zhang et al., Nature, 2015, 527, 100-104).

PTEN is expressed preferentially in the neurons in adult brains, and plays a critical role in controlling the regeneration of corticospinal neurons via downregulation of mammalian mTOR activity. The mTOR activity is profoundly suppressed in axotomized adult neurons, limiting new protein synthesis required for sustained axon regeneration. Several publications mention the involvement of PTEN in suppression of nerve regeneration and others showed the positive effect of PTEN depletion on conditions related to axon damage or impairment. Effective inhibition of PTEN would be a candidate for increasing mTOR thereby promoting nerve regeneration.

WO 2009/117389 describes the therapeutic use of inhibitors of PTEN to treat neurodegenerative disorders. WO 2015/066701 describes a method of regeneration of nerve or attenuating degeneration of nerve by administering PTEN inhibiting peptide at or near the injured nerve. WO 2011/044701 describes particular PTEN inhibiting peptides and their use in treating diseases associated with cytotoxic stress including diseases and injuries of the central nervous system.

There is a vast number of vehicles suggested as useful for delivering siRNA molecules including liposomes, protein particles, micelles, and lipid particles among others. Rungta et al., (Molecular Therapy-Nucleic Acids, 2013, 2, e136) showed that siRNA in lipid nanoparticles (LNP) may efficiently silence neuronal genes expression.

Extracellular vesicles (EVs) are membrane vesicles secreted by different types of cells. EVs are present in the blood circulation under normal physiological conditions and their levels increased in a variety of diseases such as diabetes and related vascular complications, cardiovascular disease, hematologic malignancies as well as in solid tumors.

EVs can be divided into three subpopulations: (I) exosomes: typically having a diameter of 30-100 nm in and derived from endosomal compartments; (II) microvesicles: having a diameter of 100 nm-1 μm which are released from the cell surface via “vesiculation”; and (III) apoptotic bodies: having a diameter of 1-5 μm and which are released from apoptotic cells. EVs contain several elements of the parent cell including proteins, DNA fragments, micro RNAs and mRNA.

EP 2254586 is directed to exosomes isolated from mesenchymal stem cells, said exosomes comprising at least one biological property of a mesenchymal stem cell. In addition, exosomes were proposed as carriers for different drugs including small molecules and non-coding RNA (e.g., US 2017/0247708 and Ha et al., Acta Pharmaceutica Sinica B 2016; 6 (4): 287-296). Typically, siRNA is introduced into exosomes by electroporation. WO2018/033911 to some of the inventors of the present application teaches mesenchymal stem cell derived exosomes for the treatment of neurological disorders.

WO 2019/186558 discloses pharmaceutical compositions comprising membrane vesicles, including extracellular vesicles including those referred to as exosomes, loaded with an exogenous phosphatase and tensin homolog (PTEN) inhibitor and methods of treating neurological diseases, disorders or conditions using the extracellular vesicles.

Nevertheless, there remains an unmet need for the development of additional safe, efficient, and convenient methods for treating SCI.

### SUMMARY OF THE INVENTION

The present invention discloses novel siRNA sequences that are superior in targeting and inhibiting PTEN mRNA and decreasing PTEN protein expression over the currently known sequences. The present invention relates to novel compositions and methods to enable regeneration and recovery of damaged neurons in the CNS. One example of such CNS damage is spinal cord injury (SCI). By using the siRNA molecules of the present invention, it is possible to reduce PTEN protein expression and thus regain regeneration capabilities of neurons and the entire CNS.

In one aspect, the present invention provides an RNA interference (RNAi) oligonucleotide comprising a guide strand comprising a nucleic acid sequence selected from SEQ ID NO: 1-23, for inhibiting the expression of phosphatase and tensin homolog (PTEN) protein. According to some embodiments, the RNAi is selected from siRNA and shRNA. Thus, according to some embodiments, the present invention provides an RNA interference (RNAi) oligonucleotide for inhibiting expression of PTEN comprising a guide strand comprising a nucleic acid sequence selected from SEQ ID NO: 1-23, wherein the RNAi is selected from siRNA and shRNA. According to some embodiments, the RNAi is a siRNA and the guide strand consists of a nucleic acid sequence selected from SEQ ID NO: 1-23. According to some embodiments, the RNAi oligonucleotide comprises a strand complementary to said guide strand, wherein the complementary strand is complementary to at least 14, 15, 16, 17, 18, 19, 20 or 21 contiguous nucleotides of the guide strand. According to some embodiments, the complementary strand comprises from 14 to 19 nucleotides. According to some embodiments, the complementary strand comprises a nucleic acid sequence selected from SEQ ID NO: 24-46. According to some embodiments, the RNAi is a siRNA and the guide strand comprises or consists of a nucleic acid sequence selected from SEQ ID NO: 2, 3, and 4. According to some embodiments, the RNAi is a siRNA and the sense strand comprises or consists of a nucleic acid sequence selected from SEQ ID NO: 25, 26, and 27. According to some embodiments, the RNAi oligonucleotide is siRNA comprising a guide strand comprising or consisting of the nucleic acid sequence AUCUAUAAUGAUCAGGUUCAU (SEQ ID NO: 3) and a sense strand comprising or consisting of the nucleic acid sequence GAACCUGAUCAUUAUAGAU (SEQ ID NO: 26).

According to some embodiments, the present invention provides a conjugate of the RNAi oligonucleotides as defined above. According to some embodiments, the RNAi is conjugated with a hydrophobic molecule. According to some embodiments, the hydrophobic molecule is selected from the group consisting of a sterol, a ganglioside, a lipid, a vitamin, a fatty acid, a hydrophobic peptide, and a combination thereof.

According to another aspect, the present invention provides isolated extracellular vesicles (EVs) comprising with RNAi oligonucleotides described herein, capable of inhibiting the expression of PTEN. According to some embodiments, the isolated EVs are selected from exosomes, microvesicles and a combination thereof.

According to yet another aspect, the present invention provides a pharmaceutical composition comprising at least one RNAi oligonucleotide as defined above and/or EV comprising the at least one RNAi oligonucleotide, and a pharmaceutically acceptable excipient and/or carrier. According to some embodiments, the pharmaceutical composition of the present invention is formulated for administration via an administration route selected from intranasal, intra-lesion, intrathecal, intravenous, intramuscular, subcutaneous, sublingual, oral, and intracerebral administration route. According to some embodiments, the pharmaceutical composition of the present invention is for use in regenerative therapy. According to some embodiments, the pharmaceutical composition of the present invention is for use in treating degenerative diseases or disorders. According to some embodiments, the degenerative diseases or disorders neurodegenerative disease, neuronal disorder, neuronal injury or CNS damage in a subject. According to one embodiment, the neuronal injury or damage is a spinal cord injury (SCI). According to one embodiment, the use comprises intranasal administration of the composition. According to one embodiment, the use comprises local administration of the composition.

According to another aspect, the present invention provides a method of treating a disease or condition associated with cell degeneration or death such as degenerative disease or condition, neurodegenerative disease or condition, neuronal injury or damage in a subject in need thereof, comprising administering to the subject a therapeutically effective amount of the RNAi oligonucleotides, the EVs and/or a pharmaceutical composition comprising said RNAi oligonucleotides and/or EVs.

### BRIEF DESCRIPTION OF THE FIGURES

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 shows the effect of PTEN siRNA on PTEN gene expression in SH-SY5Y cells.

FIG. 2 shows the effect of siRNA_1962 on PTEN RNA (FIG. 2A) and protein levels (FIG. 2B) in HEK293 cells (qPCR results of PTEN relative RNA levels in HEK293 cells transfected with siRNA-PTEN (20 nM, 72 hrs) compared to Non-Targeting Control (NTC)—a sample with lipofectamine with a non-targeting siRNA). Also shows a comparison of the effect with that of a commercially available siRNA denoted as POC and Non-Targeting Control (NTC) (FIGS. 2C and 2D, showing different statistical calculations). In the figures: NTC—Non-Targeting Control; POC—commercially available siRNA (see the sequences in Example 1); siPTEN=NUR001—siRNA_1962; siPTEN-chol=NUR002—siRNA_1962 conjugated with cholesterol; siPTEN-chol-cy3=NUR003—siRNA_1962 conjugated with cholesterol-cy3; FIG. 2E shows a Western blot and FIG. 2F shows densitometry analysis of PTEN protein levels in HEK293 cells transfected with siRNA_1962 (10 nM, 96 hrs) and proper controls. As seen in the figure, siRNA_1962 decreased PTEN protein levels by 90%. The experiment was performed at least twice with duplicates. n=4. * p<0.05.

FIG. 3 shows the effect of siRNA on recovery of sensory response tested by Von Frey filaments in injured rats during the weeks following treatment

FIG. 4 shows the effect of siRNA on the recovery of tail and paw pinch reflexes in injured rats after 2 weeks of treatment.

FIG. 5 shows the self-eating tendency of rats from the Exo-PTEN_chol treatment group and its controls after surgery shown in a Kaplan-Meier survival curve. Log-rank (Mantel-Cox) test with multiple comparisons Bonferroni-corrected threshold for 3 comparisons (Exo-PTEN_chol treatment group to each of the other groups, Bonferroni-corrected α value=0.166). *p value=0.01 between Exo-PTEN_chol and PTEN_chol, **p value=0.0033 between Exo-PTEN_chol and control.

FIG. 6 shows the ratio of caudal to rostral area calculated from MRI images in injured rats 10 weeks following treatment.

### DETAILED DESCRIPTION OF THE INVENTION

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

According to one aspect, the present invention provides RNA interference (RNAi) oligonucleotides comprising a guide strand comprising a nucleic acid sequence selected from SEQ ID NO: 1-23. According to some embodiments, the RNAi inhibits the expression of phosphatase and tensin homolog (PTEN). According to some embodiments, inhibiting the expression comprises inhibiting the expression of the protein. Thus, in some embodiments, the present invention provides RNA interference (RNAi) oligonucleotides comprising a guide strand comprising a nucleic acid sequence selected from SEQ ID NO: 1-23, wherein the RNAi inhibits the expression of phosphatase and tensin homolog (PTEN) protein. According to some embodiments, the RNAi oligonucleotides are selected from siRNA and shRNA. Thus, according to some embodiments, the present invention provides RNAi oligonucleotide selected from siRNA and shRNA comprising a guide strand comprising a nucleic acid sequence selected from SEQ ID NO: 1-23. According to some embodiments, the present invention provides RNAi oligonucleotide selected from siRNA and shRNA comprising a guide strand comprising a nucleic acid sequence selected from SEQ ID NO: 1-23, for inhibiting the expression of PTEN. According to some embodiments, the present invention provides RNAi oligonucleotide selected from siRNA and shRNA comprising a guide strand comprising a nucleic acid sequence selected from SEQ ID NO: 1-23, for inhibiting the expression of PTEN protein. The nucleic sequences of the sense and antisense sequences of the RNAi oligonucleotides of the present invention are summarized in Table 1.

The terms “phosphatase and tensin homolog” and “PTEN” are used herein interchangeably and refer to human phosphatase and tensin homolog enzyme being the product of EntrezGene ID: 5728 and which may have an amino acid sequence composition corresponding to UniProt Accession: P60484. Other non-human homologs are easily identified using known methods, for instance, BLAST search, and are considered to be within the scope of the invention. PTEN protein acts as a phosphatase to dephosphorylate phosphatidylinositol (3,4,5)-trisphosphate (PtdIns (3,4,5)P3 or PIP3). PTEN specifically catalyzes the dephosphorylation of the 3′ phosphate of the inositol ring in PIP3, resulting in the biphosphate product PIP2 (PtdIns (4,5)P2). This dephosphorylation is important because it results in inhibition of the AKT signaling pathway.

The term “polynucleotide” as used herein refers to a long nucleic acid comprising more than 150 nucleotides. The term “oligonucleotide” as used herein refers to a short single-stranded or double-stranded sequence of nucleic acid such as ribonucleic acid (RNA), deoxyribonucleic acid (DNA) or mimetics thereof, said nucleic acid has typically less than or equal to 150 nucleotides. According to some embodiments, the oligonucleotide consists of 2 to 150, 10 to 100, or 14 to 50 nucleotides. According to other embodiments, the oligonucleotide consists of from 14 to 40, from 17 to 35, or from 18 to 30 nucleic acids.

As used herein, the term “RNA silencing” refers to a group of regulatory mechanisms (e.g., RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

As used herein, the terms “RNA silencing agent”, “RNA silencing molecule” and “RNA silencing oligonucleotide” are used herein interchangeably and refer to an RNA that is capable of inhibiting or “silencing” the expression of a target gene. In certain embodiments, the RNA silencing agent is capable of preventing complete processing (e.g., the full translation and/or expression) of an mRNA molecule through a post-transcriptional silencing mechanism, e.g., by degradation of mRNA via RNA interference. RNA silencing agents include noncoding RNA molecules, for example, RNA duplexes comprising paired strands, as well as precursor RNAs from which such small non-coding RNAs can be generated. Exemplary RNA silencing agents, referred also as RNA interference oligonucleotides, include dsRNAs such as siRNAs, miRNAs, and shRNAs. In one embodiment, the RNA silencing agent is capable of inducing RNA interference. In another embodiment, the RNA silencing agent is capable of mediating translational repression.

The term “RNA interference” refers to the process of sequence-specific post-transcriptional gene silencing in animals mediated by RNA interference oligonucleotides such as short interfering RNAs (siRNAs) and shRNAs. The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla. Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA.

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

The term “small interfering RNA” and “siRNA” refer to small inhibitory RNA duplexes (generally between 18-30 base-pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand. This can be attributed to asymmetrical strand loading into RISC, as the opposite efficacy patterns are observed when targeting the antisense transcript.

According to some embodiments, the RNAi is siRNA. According to some embodiments, the siRNA inhibiting expression of PTEN comprises a guide strand comprising a nucleic acid sequence selected from SEQ ID NO: 1-23. According to some embodiments, the siRNA comprises a guide strand consisting of a nucleic acid sequence selected from SEQ ID NO: 1-23.

The terms “guide strand”, “guide strand oligonucleotide”, and “antisense strand” are used herein interchangeably and refer to a stand of siRNA or shRNA that is complementary to a sequence within an mRNA molecule.

As used herein the term “inhibiting expression of PTEN” has the meaning of inhibiting the expression of PTEN gene and inhibiting the production of PTEN protein.

According to other embodiments, the RNAi is shRNA. According to some embodiments, the shRNA inhibiting expression of PTEN comprises a nucleic acid sequence selected from SEQ ID NO: 1-23.

The term “small hairpin RNA” and “shRNA”, as used herein, refers to an RNA agent having a stem-loop structure, as known in art, comprising a first and second region of the complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Typically the shRNA molecule has less than approximately 400 to 500 nucleotides (nt), or less than 100 to 200 nt, in which at least one stretch of at least 14 to 100 nucleotides (e.g., 17 to 50 nt, 19 to 29 nt) is based paired with a complementary sequence located on the same RNA molecule (single RNA strand), and where said sequence and complementary sequence are separated by an unpaired region of at least about 4 to 7 nucleotides (or about 9 to about 15 nt, about 15 to about 100 nt, about 100 to about 1000 nt) which forms a single-stranded loop above the stem structure created by the two regions of base complementarity.

According to some embodiments, the RNAi oligonucleotide, such as siRNA or shRNA, is not natural, i.e., does not exist in nature and is being artificially designed modified and/or manufactured.

According to some embodiments, the RNAi oligonucleotide of the present invention, e.g., siRNA or shRNA comprises a sense strand, i.e., a strand complementary to a guide strand. According to some embodiments, wherein the complementary strand is complementary to at least 14, 15, 16, 17, 18, 19, 20 or 21 contiguous nucleotides of the guide strand. According to some embodiments, the complementary strand is complementary to from 14 to 19 contiguous nucleotides of the guide strand. According to some embodiments, the sense strand comprises from 14 to 19 nucleotides. According to some embodiments, the complementary strand comprises 14, 15, 16, 17, 18 or 19 nucleotides. According to some embodiments, the complementary strand consists of 14, 15, 16, 17, 18 or 19 nucleotides. According to some embodiments, the complementary strand consists of and is complementary to 14, 15, 16, 17, 18 or 19 contiguous nucleotides of the guide strand. According to some embodiments, the complementary strand comprises a nucleic acid sequence selected from SEQ ID NO: 24-46.

According to some embodiments, the RNAi oligonucleotide inhibiting expression of PTEN is siRNA comprising a guide strand comprising a nucleic acid sequence selected from SEQ ID NO: 1-23. According to some embodiments, the RNAi oligonucleotide inhibiting expression of PTEN is siRNA comprising a guide strand consisting of a nucleic acid sequence selected from SEQ ID NO: 1-23. According to some embodiments, the RNAi oligonucleotide inhibiting expression of PTEN is siRNA comprising a guide strand consisting of a nucleic acid sequence selected from SEQ ID NO: 1-23. According to some embodiments, the siRNA comprises a guide strand comprising or consisting of a nucleic acid sequence AUCUAUAAUGAUCAGGUUCAU (SEQ ID NO: 3). According to some embodiments, the siRNA comprises a guide strand comprising or consisting of a nucleic acid sequence UUCCGCCACUGAACAUUGGAA (SEQ ID NO: 2). According to some embodiments, the siRNA comprises a guide strand comprising or consisting of a nucleic acid sequence AAGUUCCGCCACUGAACAUUG (SEQ ID NO: 4). According to some embodiments, the siRNA comprises a guide strand comprising or consisting of a nucleic acid sequence selected from SEQ ID NO: 2-4. According to some embodiments, the siRNA comprises a guide strand comprising or consisting of a nucleic acid sequence selected from SEQ ID NO: 1-5. According to another embodiment, the siRNA inhibiting expression of PTEN comprises a sense strand complementary to the guide strand and comprises a nucleic acid sequence selected from SEQ ID NO: 24-46. According to another embodiment, the siRNA inhibiting expression of PTEN comprises a sense strand complementary to the guide strand and consisting of a nucleic acid sequence selected from SEQ ID NO: 24-46. According to another embodiment, the siRNA inhibiting expression of PTEN comprises a sense strand complementary to the guide strand and comprising or consisting of a nucleic acid sequence selected from SEQ ID NO: 25-27. According to some embodiments, the siRNA inhibiting expression of PTEN comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii) SEQ ID NO: 22 and 45; or (xxiii) SEQ ID NO: 23 and 46.

According to some embodiments, the siRNA of the present invention comprises a guide strand and a sense strand comprising the nucleic acid sequences SEQ ID NO: 1 and SEQ ID NO: 24, respectively. According to some embodiments, the siRNA of the present invention comprises a guide strand and a sense strand consisting of the nucleic acid sequences SEQ ID NO: 1 and SEQ ID NO: 24, respectively.

According to some embodiments, the siRNA of the present invention comprises a guide strand and a sense strand comprising the nucleic acid sequences SEQ ID NO: 2 and SEQ ID NO: 25, respectively. According to some embodiments, the siRNA of the present invention comprises a guide strand and a sense strand consisting of the nucleic acid sequences SEQ ID NO: 2 and SEQ ID NO: 25, respectively. In some examples, the siRNA is denoted as siRNA_1455.

According to some embodiments, the siRNA of the present invention comprises a guide strand and a sense comprising strand the nucleic acid sequences AUCUAUAAUGAUCAGGUUCAU (SEQ ID NO: 3) and GAACCUGAUCAUUAUAGAU (SEQ ID NO: 26), respectively. According to some embodiments, the siRNA of the present invention comprises a guide strand and a sense strand consisting of the nucleic acid sequences AUCUAUAAUGAUCAGGUUCAU (SEQ ID NO: 3) and GAACCUGAUCAUUAUAGAU (SEQ ID NO: 26), respectively. In some examples, the siRNA is denoted as siRNA_1962.

According to some embodiments, the siRNA of the present invention comprises a guide strand and a sense strand comprising the nucleic acid sequences SEQ ID NO: 4 and SEQ ID NO: 27, respectively. According to some embodiments, the siRNA of the present invention comprises a guide strand and a sense strand consisting of the nucleic acid sequences SEQ ID NO: 4 and SEQ ID NO: 27, respectively. In some examples, the siRNA is denoted as siRNA_1458.

According to some embodiments, the siRNA of the present invention comprises a guide strand and a sense strand comprising the nucleic acid sequences SEQ ID NO: 5 and SEQ ID NO: 28, respectively. According to some embodiments, the siRNA of the present invention comprises a guide strand and a sense strand consisting of the nucleic acid sequences SEQ ID NO: 5 and SEQ ID NO: 28, respectively. According to some embodiments, the siRNA of the present invention comprises a guide strand and a sense strand comprising or consisting of the nucleic acid sequences SEQ ID NO: 6 and SEQ ID NO: 29, respectively. According to some embodiments, the siRNA of the present invention comprises a guide strand and a sense strand comprising or consisting of the nucleic acid sequences SEQ ID NO: 7 and SEQ ID NO: 30, respectively. According to some embodiments, the siRNA of the present invention comprises a guide strand and a sense strand comprising or consisting of the nucleic acid sequences SEQ ID NO: 8 and SEQ ID NO: 31, respectively. According to some embodiments, the siRNA of the present invention comprises a guide strand and a sense strand comprising or consisting of the nucleic acid sequences SEQ ID NO: 9 and SEQ ID NO: 32, respectively. According to some embodiments, the siRNA of the present invention comprises a guide strand and a sense strand comprising or consisting of the nucleic acid sequences SEQ ID NO: 10 and 33, respectively. According to some embodiments, the siRNA of the present invention comprises a guide strand and a sense strand comprising or consisting of the nucleic acid sequences SEQ ID NO: 11 and 34, respectively. According to some embodiments, the siRNA of the present invention comprises a guide strand and a sense strand comprising or consisting of the nucleic acid sequences SEQ ID NO: 12 and 35, respectively. According to some embodiments, the siRNA of the present invention comprises a guide strand and a sense strand comprising or consisting of the nucleic acid sequences SEQ ID NO: 13 and 36, respectively. According to some embodiments, the siRNA of the present invention comprises a guide strand and a sense strand comprising or consisting of the nucleic acid sequences SEQ ID NO: 14 and 37, respectively. According to some embodiments, the siRNA of the present invention comprises a guide strand and a sense strand comprising or consisting of the nucleic acid sequences SEQ ID NO: 15 and 38, respectively. According to some embodiments, the siRNA of the present invention comprises a guide strand and a sense strand comprising or consisting of the nucleic acid sequences SEQ ID NO: 16 and 39, respectively. According to some embodiments, the siRNA of the present invention comprises a guide strand and a sense strand comprising or consisting of the nucleic acid sequences SEQ ID NO: 17 and 40, respectively. According to some embodiments, the siRNA of the present invention comprises a guide strand and a sense strand comprising or consisting of the nucleic acid sequences SEQ ID NO: 18 and 41, respectively. According to some embodiments, the siRNA of the present invention comprises a guide strand and a sense strand comprising or consisting of the nucleic acid sequences SEQ ID NO: 19 and 42, According to some embodiments, the siRNA of the present invention comprises a guide strand and a sense strand comprising or consisting of the nucleic acid sequences SEQ ID NO: 20 and 43, respectively. According to some embodiments, the siRNA of the present invention comprises a guide strand and a sense strand comprising or consisting of the nucleic acid sequences SEQ ID NO: 21 and 44, respectively. According to some embodiments, the siRNA of the present invention comprises a guide strand and a sense strand comprising or consisting of the nucleic acid sequences SEQ ID NO: 22 and 45, respectively. According to some embodiments, the siRNA of the present invention comprises a guide strand and a sense strand comprising or consisting of the nucleic acid sequences SEQ ID NO: 23 and 46, respectively.

According to some embodiments, the RNAi oligonucleotide inhibiting expression of PTEN is shRNA comprising a guide strand comprising a nucleic acid sequence selected from SEQ ID NO: 1-23. According to some embodiments, the RNAi oligonucleotide inhibiting expression of PTEN is shRNA comprising a guide strand consisting of a nucleic acid sequence selected from SEQ ID NO: 1-23. According to another embodiment, the shRNA inhibiting expression of PTEN comprises a sense complementary strand comprising a nucleic acid sequence selected from SEQ ID NO: 24-46. According to another embodiment, the shRNA inhibiting expression of PTEN comprises a sense strand complementary to the guide strand and consisting of a nucleic acid sequence selected from SEQ ID NO: 24-46. According to some embodiments, the shRNA inhibiting expression of PTEN comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii); SEQ ID NO: 22 and 45; or (xxiii) SEQ ID NO: 23 and 46. According to some embodiments, the pairs of sense and antisense sequences are as described for siRNA. According to some embodiments, the shRNA of the present invention comprises a guide strand and a sense strand comprising the nucleic acid sequences SEQ ID NO: 1 and SEQ ID NO: 24, respectively. According to some embodiments, the shRNA of the present invention comprises a guide strand and a sense strand consisting of the nucleic acid sequences SEQ ID NO: 1 and SEQ ID NO: 24, respectively.

According to some embodiments, the shRNA of the present invention comprises a guide strand and a sense strand comprising the nucleic acid sequences SEQ ID NO: 2 and SEQ ID NO: 25, respectively. According to some embodiments, the shRNA of the present invention comprises a guide strand and a sense strand consisting of the nucleic acid sequences SEQ ID NO: 2 and SEQ ID NO: 25, respectively.

According to some embodiments, the shRNA of the present invention comprises a guide strand and a sense strand comprising the nucleic acid sequences SEQ ID NO: 3 and SEQ ID NO: 26, respectively. According to some embodiments, the shRNA of the present invention comprises a guide strand and a sense strand consisting of the nucleic acid sequences SEQ ID NO: 3 and SEQ ID NO: 26, respectively.

According to some embodiments, the shRNA of the present invention comprises a guide strand and a sense strand comprising the nucleic acid sequences SEQ ID NO: 4 and SEQ ID NO: 27, respectively. According to some embodiments, the shRNA of the present invention comprises a guide strand and a sense strand consisting of the nucleic acid sequences SEQ ID NO: 4 and SEQ ID NO: 27, respectively.

According to some embodiments, the RNAi oligonucleotide of the present invention is conjugated to a moiety for improving at least one property such as solubility, permeability, loading capacity, stability, blood circulation term or for targeting the RNAi oligonucleotides of a specific site. In some embodiments, the moiety is heterologous and/or exogenous. The term “exogenous” refers to a molecule or substance (e.g., a compound, nucleic acid or protein) that is not normally present in a cell or the combination or conjugation is not present in a cell. Thus, according to some embodiments, the present invention provides conjugates of the RNAi oligonucleotides of the present invention. According to some embodiments, the RNAi oligonucleotide of the present invention is conjugated with a hydrophilic moiety. According to some embodiments, the RNAi oligonucleotide of the present invention is conjugated with a hydrophobic moiety. Thus, according to some embodiments, the siRNA or the shRNA oligonucleotide of the present invention is conjugated with a hydrophobic molecule. According to some embodiments, the hydrophobic molecule is bound to the guide strand. According to some embodiments, the hydrophobic molecule is bound to the complementary (sense) strand. According to some embodiments, the moiety is a loading moiety. The term “loading moiety” refers to a moiety allowing or enhancing the loading of molecules into a carrier entity such as extracellular vesicles (EVs) or liposomes.

According to one embodiment, the said hydrophobic moiety is selected from the group consisting of a sterol, a ganglioside, a lipid, a vitamin, a fatty acid, a peptide, and a combination thereof. According to one embodiment, the RNA interference oligonucleotide is conjugated with a sterol. In exemplary embodiments, the moiety is a sterol cholesterol molecule, therefore according to such embodiments, the RNA interference oligonucleotide is conjugated with a cholesterol. According to some embodiments, one of the strands of the double-stranded RNAi is conjugated with a hydrophobic molecule such as cholesterol. According to other embodiments, two strands of the double-stranded RNAi are conjugated with a hydrophobic molecule such as cholesterol. According to other embodiments, the RNA interference oligonucleotide is conjugated with a molecule selected from monosialotetrahexosylganglioside (GM1), a lipid, a vitamin, a small molecule, a peptide, or a combination thereof. In some embodiments, the moiety is a lipid. For example, in certain embodiments, the moiety is palmitoyl. In some embodiments, the moiety is a sterol, e.g., cholesterol. Additional hydrophobic moieties include, for example, phospholipids, vitamin D, vitamin E, squalene, and fatty acids. In another exemplary embodiment, the RNAi oligonucleotide is conjugated to myristic acid, or a derivative thereof (e.g., myristoylated oligonucleotide cargo). In some embodiments, the hydrophobic moiety is conjugated at the termini of the oligonucleotide cargo (i.e., “terminal modification”). In other embodiments, the hydrophobic moiety is conjugated to other portions of the oligonucleotide molecule.

According to some embodiments, the RNAi oligonucleotide of the present invention is conjugated with a hydrophobic moiety selected from the group consisting of a sterol, a ganglioside, a lipid, a vitamin, a fatty acid, a hydrophobic peptide, and a combination thereof.

According to some embodiments, the siRNA is conjugated with cholesterol. According to some embodiments, the cholesterol is conjugated to the guide strand of siRNA. According to other embodiments, the cholesterol is conjugated to the complementary strand of siRNA. According to some embodiments, the cholesterol is conjugated to shRNA. According to some embodiments, the siRNA comprises a guide strand and a sense strand comprising or consisting of the nucleic acid sequences SEQ ID NO: 2 and SEQ ID NO: 25, respectively, is conjugated with cholesterol molecule. According to some embodiments, the siRNA comprises a guide strand and a sense strand comprising or consisting of the nucleic acid sequences SEQ ID NO: 3 and SEQ ID NO: 26, respectively is conjugated with cholesterol molecule. According to some embodiments, the siRNA comprising a guide strand and a sense strand comprising or consisting of the nucleic acid sequences SEQ ID NO: 4 and SEQ ID NO: 27, respectively is conjugated with cholesterol molecule. According to some embodiments, the cholesterol is conjugated to 5′ of the oligonucleotides. According to some embodiments, the cholesterol is conjugated to 3′ of the oligonucleotides.

According to another aspect, the present invention provides a composition comprising the RNAi oligonucleotides of the present invention and a carrier. Any one of the above definitions, terms and embodiments are encompassed and apply herein as well. The term “carrier” as used herein refers to as a class any compound or composition useful in facilitating storage, stability, administration, cell targeting and/or delivery of the topical composition, including, without limitation, suitable vehicles, skin conditioning agents, skin protectants, diluents, emollients, solvents, excipients, pH modifiers, salts, colorants, rheology modifiers, thickeners, lubricants, humectants, antifoaming agents, erodeable polymers, hydrogels, surfactants, emulsifiers, emulsion stabilizers, adjuvants, surfactants, preservatives, chelating agents, fatty acids, mono- di- and tri-glycerides and derivates thereof, waxes, oils and water.

According to some embodiments, the composition is a pharmaceutical composition and the carrier is a pharmaceutically acceptable carrier. Thus, according to some embodiments, the present invention provides a pharmaceutical composition comprising a plurality of siRNA and/or shRNA of the present invention as defined above, and a pharmaceutically acceptable carrier. According to some embodiments, the siRNA comprises a guide strand and a sense strand comprising or consisting of the nucleic acid sequences SEQ ID NO: 2 and SEQ ID NO: 25, respectively, and optionally is conjugated with cholesterol molecule. According to some embodiments, the siRNA comprises a guide strand and a sense strand comprising or consisting of the nucleic acid sequences SEQ ID NO: 3 and SEQ ID NO: 26, respectively, and optionally is conjugated with cholesterol molecule. According to some embodiments, the siRNA comprises a guide strand and a sense strand comprising or consisting of the nucleic acid sequences SEQ ID NO: 4 and SEQ ID NO: 27, respectively, and optionally is conjugated with cholesterol molecule.

According to another aspect, the present invention provides isolated extracellular vesicles (EVs) comprising the RNA interference (RNAi) oligonucleotides as described in any of the above aspects and embodiments inhibiting the expression of PTEN protein. All terms, embodiments and definitions disclosed in any one of the above aspects apply and are encompassed herein as well. According to some embodiments, the RNAi oligonucleotides are loaded on/into the EVs. Thus, according to some embodiments, the present invention provides isolated extracellular vesicles (EVs) loaded with RNA interference (RNAi) oligonucleotides inhibiting the expression of PTEN. According to some embodiments, the present invention provides EVs comprising RNAi oligonucleotides inhibiting the expression of PTEN.

According to some embodiments, the RNAi oligonucleotide is exogenous. The term “exogenous” as used herein refers to a molecule or substance (e.g., a compound, nucleic acid or protein) that originates from outside of a given membrane vesicle such as EVs, and is not naturally present in the vesicle. With respect to EVs, the term refers to molecules or substances that are not naturally present in the vesicle and not in the cells from which the EVs are derived. According to some embodiments, the term “exogenous” refers to synthetic non-natural molecules. According to some embodiments, the substance is artificially loaded to the EVs or to cells from which the EVs are derived. With respect to peptides, proteins and nucleic acids the term means that the compound is artificially loaded to the EVs or to cells from which the vesicles are derived or artificially expressed within cells from which the vesicles are derived, however, the compound is not naturally expressed in the parent cells.

The terms “extracellular vesicles” and “EVs” are used herein interchangeably and refer to cell-derived vesicles comprising a membrane that encloses an internal space. Generally, EVs range in diameter from 30 nm to 1500 nm, more frequently from 40 to 1200 nm, and may comprise various cargo molecules either within the internal space, displayed on the external surface of the extracellular vesicle, and/or spanning the membrane. Said cargo molecules may comprise nucleic acids, proteins, carbohydrates, lipids, small molecules, and/or combinations thereof. EVs can be divided into three subpopulations: (I) exosomes: having a diameter of 30-150 nm in diameter and derived from endosomal compartments; (II) microvesicles: having a diameter of 100 nm-1 μm which are released from the cell surface via “vesiculation”; and (III) apoptotic bodies: having a diameter of 1-5 μm and which are released from apoptotic cells. The term EVs comprise also the terms “exosome” and “microvesicles”. The terms “exosomes” and “nanovesicle” are used herein interchangeably and refer to EVs having a size of between 30 to 150 nm in diameter. In some references, exosomes refer to EVs having a size of between 30 to 100 nm in diameter. The term “microvesicles” as used herein refers to EVs having a size of between 150 to 1000 nm in diameter. Generally, the EVs may comprise at least a part of the molecular contents of the cells from which they are originated, e.g., lipids, fatty acids, polypeptides, polynucleotides, proteins and/or saccharides.

The EVs of the present invention are mostly spherical and the terms “size”. “particle size”. “average particle size” and “particle diameter size” used herein interchangeably refer to the diameter of the EVs or to the longer diameter of the EVs. Any known method for measurement of particle size may be used to determine the size of the EVs of the present invention. A non-limiting example is nanoparticle-tracking analysis (NTA).

According to some embodiments, the isolated EVs are exosomes. According to one embodiment, the exosomes have a diameter of from 30 to 150 nm, from 40 to 120 nm, from 50 to 100 nm, from 30 to 100 nm, from 30 nm to 80 nm, or from 60 nm to 80 nm.

According to another embodiment, the EVs are microvesicles. According to one embodiment, the microvesicles have a diameter of from 100 to 1000 nm, from 120 to 800 nm, from 150 to 600 nm or from 200 to 400 nm. According to another embodiment, the microvesicles have a size of 100 to 300 nm or 150 to 250 nm.

According to some embodiments, the EVs have a diameter from 30 to 250 nm or from 50 to 200 nm. According to some embodiments, the EVs have a diameter from 70 to 170 nm or from 80 to 150 nm.

The EVs may have a range of sizes, such as between 2 nm to 20 nm, 2 nm to 50 nm, 2 nm to 100 nm. 2 nm to 150 nm or 2 nm to 200 nm. The EVs may have a size between 20 nm to 50 nm, 20 nm to 100 nm. 20 nm to 150 nm or 20 nm to 200 nm. The EVs may have a size between 50 nm to 100 nm, 50 nm to 150 nm or 50 nm to 200 nm. The EVs may have a size between 100 nm to 150 nm or 100 nm to 200 nm. The EVs may have a size between 150 nm to 200 nm. The EVs may have a size of 100 to 600 nm, 150 to 500 nm, or 200 to 400 nm.

The size may be determined by various means. In principle, the size may be determined by size fractionation and filtration through a membrane with the relevant size cut-off.

According to a further embodiment, the isolated EVs are a combination of small and large vesicles, e.g., of microvesicles and exosomes.

As described hereinabove, the EVs are derived from cells. The terms “derived from” and “originated from” are used herein interchangeably and refer to vesicles that are produced within, by, or from, a specified cell, cell type, or population of cells. As used herein, the terms “parent cell”, “producer cell” and “original cell” include any cell from which the extracellular vesicle is derived and isolated. The term also encompasses a cell that shares a protein, lipid, sugar, or nucleic acid component of the extracellular vesicle. For example, a “parent cell” or “producer cell” includes a cell that serves as a source for the extracellular vesicle. According to some embodiments, the cells are eukaryotic cells.

The EVs may be derived from biological cells by any of several means, for example by secretion, budding or dispersal from the biological cells. The EVs may be something that is isolatable from a mesenchymal stem cell (MSC), neural crest cell (NCC), mesenchymal stem cell conditioned medium (MSC-CM) or neural crest cell conditioned medium. The EVs may be responsible for or have at least an activity of the parent cells such as of MSC, NCC, NCC-CM or MSC-CM. The EVs may be responsible for, and carry out, substantially most or all of the functions of the activity of the parent cells such as of MSC, NCC, NCC-CM or MSC-CM. For example, the EVs may be a substitute (or biological substitute) for the MSC, NCC, NCC-CM or MSC-CM. For example, the EVs may be produced, exuded, emitted or shed from the biological cells. Where the biological cell is in cell culture, the particle may be secreted into the cell culture medium.

Examples of biological cells from which the EVs may be derived include, adherent cells which express mesenchymal markers such as mesenchymal stem cells, oral mucosa stem cells or olfactory ensheathing cells, astrocytes, and neural crest cells. Thus, according to some embodiments, the present invention provides a pharmaceutical composition comprising EVs loaded with an exogenous PTEN inhibitor, wherein the EVs are derived from adherent cells expressing mesenchymal markers. According to one embodiment, the adherent cells expressing mesenchymal markers are selected from mesenchymal stem cells (MSC), oral mucosa stem cells and olfactory ensheathing cells. According to one embodiment, the cells are mesenchymal stem cells (MSC).

The term “mesenchymal stem cells” refers to multipotent stromal cells that can differentiate into a variety of cell types, as well known in the art, including to: osteoblasts (bone cells), chondrocytes (cartilage cells), myocytes (muscle cells) and adipocytes (fat cells).

In their pluripotent state, mesenchymal stem cells typically express the following markers: CD105, CD166, CD29, CD90, and CD73, and do not express CD34, CD45 and CD133.

Mesenchymal stem cells may be isolated from a variety of tissues including but not limited to bone marrow, adipose tissue, dental pulp, oral mucosa, peripheral blood and amniotic fluid. According to one embodiment, the mesenchymal stem cells are isolated from to bone marrow. According to one embodiment, the mesenchymal stem cells are originated from a site selected from bone marrow, adipose tissue, umbilical cord, dental pulp, oral mucosa, peripheral blood and amniotic fluid. According to some embodiments, the EVs are derived from bone marrow originated MSC. According to other embodiments, the EVs are derived from the adipose tissue originated MSC. According to some such embodiments, the EVs are selected from exosomes, microvesicles and a combination thereof. According to some embodiments, the cells express CD105, CD166, CD29, CD90, and CD73 markers. According a further embodiment, the cells express CD105, CD166, CD29, CD90, and CD73, and do not express CD34, CD45 and CD133. According to some embodiments, the cells are selected from dental pulp stem cells (DPSCs), exfoliated deciduous teeth stem cells (SHED), periodontal ligament stem cells (PDLSCs), apical papilla stem cells (SCAP) and dental follicle progenitor cells (DFPCs).

According to some such embodiments, the EVs comprise or express at least a fraction of the markers expressed by the cell from which EVs are derived.

The EVs may comprise one or more proteins, oligonucleotides or polynucleotides secreted by a particular cell type e.g., mesenchymal stem cell or neural crest cell. The EVs may comprise one or more proteins or polynucleotides present in mesenchymal stem cell conditioned medium (MSC-CM). In a particular embodiment, the EVs may comprise miRNAs which are derived from MSCs or neural crest cells. For example, the EVs may comprise 10% or more, 20% or more, 30% or more, 40% or more, 50% or more, 60% or more or 70% or more of these proteins and/or polynucleotides. The EVs may comprise substantially about 75% of these proteins and/or polynucleotides. The proteins may be defined by reference to a list of proteins or gene products of a list of genes.

The EVs may have at least one property of a mesenchymal stem cell. The particle may have a biological property, such as biological activity. The particle may have any of the biological activities of an MSC. The particle may for example have a therapeutic or restorative activity of an MSC.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46 (12): 3349-60.

Mesenchymal stem cell cultures may be generated by diluting BM aspirates (usually 20 ml) with equal volumes of Hank's balanced salt solution (HBSS; GIBCO Laboratories, Grand Island, NY, USA) and layering the diluted cells over about 10 ml of a Ficoll column (Ficoll-Paque; Pharmacia, Piscataway, NJ, USA). Following 30 minutes of centrifugation at 2,500×g, the mononuclear cell layer is removed from the interface and suspended in HBSS. Cells are then centrifuged at 1,500×g for 15 minutes and resuspended in a complete medium (MEM, a medium without deoxyribonucleotides or ribonucleotides; GIBCO); 20% fetal calf serum (FCS) derived from a lot selected for the rapid growth of MSCs (Atlanta Biologicals, Norcross, GA); 100 units/ml penicillin (GIBCO), 100 μg/ml streptomycin (GIBCO); and 2 mM L-glutamine (GIBCO). Resuspended cells are plated in about 25 ml of medium in a 10 cm culture dish (Corning Glass Works, Corning, NY) and incubated at 37° C. with 5% humidified CO₂. Following 24 hours in culture, nonadherent cells are discarded, and the adherent cells are thoroughly washed twice with phosphate-buffered saline (PBS). The medium is replaced with a fresh complete medium every 3 or 4 days for about 14 days. Adherent cells are then harvested with 0.25% trypsin and 1 mM EDTA (Trypsin/EDTA, GIBCO) for 5 min at 37° C., replated in a 6-cm plate and cultured for another 14 days. Cells are then trypsinized and counted using a cell counting device such as for example, a hemocytometer (Hausser Scientific, Horsham, PA). Cultured cells are recovered by centrifugation and resuspended with 5% DMSO and 30% FCS at a concentration of 1 to 2×10⁶ cells per ml. Aliquots of about 1 ml each are slowly frozen and stored in liquid nitrogen.

To expand the mesenchymal stem cell fraction, frozen cells are thawed at 37° C., diluted with a complete medium and recovered by centrifugation to remove the DMSO. Cells are resuspended in a complete medium and plated at a concentration of about 5,000 cells/cm². Following 24 hours in culture, nonadherent cells are removed and the adherent cells are harvested using Trypsin/EDTA, dissociated by passage through a narrowed Pasteur pipette, and preferably replated at a density of about 1.5 to about 3.0 cells/cm². Under these conditions, MSC cultures can grow for about 50 population doublings and be expanded for about 2000-fold (Colter DC., et al., Proc Natl Acad Sci USA. 97:3213-3218, 2000).

MSC cultures utilized by some embodiments of the invention include three groups of cells which are defined by their morphological features: small and agranular cells (referred to as RS-1, hereinbelow), small and granular cells (referred to as RS-2, herein below) and large and moderately granular cells (referred to as mature MSCs, herein below). The presence and concentration of such cells in culture can be assayed by identifying the presence or absence of various cell surface markers, by using, for example, immunofluorescence, in situ hybridization, and activity assays.

According to a particular embodiment, the EVs are derived from cells expressing markers from neural crest cells. According to a particular embodiment, the EVs are derived from neural crest cells. According to another embodiment, the neural crest cells are cranial neural crest cells. According to some embodiments, the cranial neural crest cells include, but are not limited to dental pulp stem cells (DPSCs), exfoliated deciduous teeth stem cells (SHED), periodontal ligament stem cells (PDLSCs), apical papilla stem cells (SCAP) and dental follicle progenitor cells (DFPCs). According to some embodiments, such cells express mesenchymal markers, as defined above.

The EVs may be produced or isolated in several ways. Such a method may comprise isolating the EVs from mesenchymal stem cells (MSC) or from neural crest cells (NCC).

Therefore, the EVs of the present invention are isolated EVs.

The terms “purify”, “purified,” “purifying”, “isolate”, “isolated,” and “isolating” are used herein interchangeably and refer to the state of a population (e.g., a plurality of known or unknown amounts and/or concentration) of EVs, that have undergone one or more processes of purification/isolation, e.g., a selection of the desired EVs, or alternatively a removal or reduction of residual biological products and/or removal of undesirable EVs, e.g., removing EVs having a particular size. According to one embodiment, the ratio of EVs to residual parent cells is at least 2, 3, 4, 5, 6, 8 or 10 times higher, or in certain advantageous embodiments at least 50, 100, 1000, or 2000 times higher than in the initial material. According to some embodiments, the ratio is the weight ratio. In some advantageous embodiments, the term “isolated” has the meaning of substantially cell-free or cell-free and may be substituted by it.

According to some embodiments, the EVs, e.g., exosomes are derived from adherent cells expressing mesenchymal markers. According to some embodiments, the adherent cells expressing mesenchymal markers are selected from mesenchymal stem cells (MSC) and olfactory ensheathing cells.

The EVs may be produced or isolated in several ways. Such a method may comprise isolating the EVs from mesenchymal stem cells (MSC) or from neural crest cells (NCC).

According to some embodiments, the present invention provides isolated EVs loaded with RNA interference (RNAi) oligonucleotides inhibiting the expression of PTEN as described in any one of the above embodiments and aspects. According to some embodiments, the loading may be performed according to any known method. According to some embodiments, the loading is done ex vivo. According to some embodiments, the RNAi oligonucleotide is as defined in any one of the above embodiments. According to some embodiments, the RNAi oligonucleotide is selected from siRNA and shRNA. According to some embodiments, the RNAi oligonucleotide is siRNA. According to some embodiments, the siRNA or shRNA inhibiting the expression of PTEN comprises a guide strand comprising a nucleic acid sequence selected from SEQ ID NO: 1-23. According to some embodiments, the siRNA or shRNA comprises a guide strand consisting of a nucleic acid sequence selected from SEQ ID NO: 1-23. According to some embodiments, the siRNA comprises a sense (complementary) strand, i.e., a strand complementary to said guide strand. According to some embodiments, wherein the complementary strand is complementary to at least 14, 15, 16, 17, 18, 19, 20 or 21 contiguous nucleotides of the guide strand. According to some embodiments, the complementary strand is complementary to from 14 to 19 contiguous nucleotides of the guide strand. According to some embodiments, the complementary strand comprises from 14 to 19 nucleotides. According to some embodiments, the complementary strand comprises from 14, 15, 16, 17, 18 or 19 nucleotides. According to some embodiments, the complementary strand comprises a nucleic acid sequence selected from SEQ ID NO: 24-46. According to one embodiment, siRNA or siRNA is conjugated with a hydrophobic moiety. According to some embodiments, the hydrophobic moiety is selected from the group consisting of a sterol, a ganglioside, a lipid, a vitamin, a fatty acid, a peptide, and a combination thereof. According to one embodiment, the siRNA or shRNA is conjugated with a sterol. In exemplary embodiments, the moiety is a sterol cholesterol molecule, therefore according to such embodiments, the siRNA or shRNA is conjugated with a cholesterol.

According to some embodiments, the RNAi oligonucleotide inhibiting the expression of PTEN is siRNA. According to some embodiments, the present invention provides isolated EVs comprising with siRNA inhibiting the expression of PTEN comprising a guide strand comprising or consisting of a nucleic acid sequence selected from SEQ ID NO: 1-23. According to another embodiment, the siRNA comprises a complementary strand comprising a nucleic acid sequence selected from SEQ ID NO: 24-46. According to some embodiments, the siRNA inhibiting the expression of PTEN comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii); SEQ ID NO: 22 and 45; or (xxiii) SEQ ID NO: 23 and 46. According to some embodiments, the siRNA comprises a guide strand and a sense strand comprising or consisting of the nucleic acid sequences SEQ ID NO: 2 and SEQ ID NO: 25, respectively. According to some embodiments, the siRNA comprises a guide strand and a sense strand comprising or consisting of the nucleic acid sequences SEQ ID NO: 3 and SEQ ID NO: 26, respectively. According to some embodiments, the siRNA comprises a guide strand and a sense strand comprising or consisting of the nucleic acid sequences SEQ ID NO: 4 and SEQ ID NO: 27, respectively. According to some embodiments, the siRNA is conjugated with cholesterol. According to some embodiments, the EVs are exosomes, microvesicles or a combination thereof. According to some embodiments, the EVs are derived from mesenchymal stem cells. According to some embodiments, the EVs are derived from bone marrow mesenchymal stem cells. According to some embodiments, the EVs are exosomes.

According to some embodiments, the RNAi oligonucleotide inhibiting the expression of PTEN is shRNA. According to some embodiments, the present invention provides isolated EVs loaded with shRNA inhibiting the expression of PTEN comprising a guide strand comprising or consisting of a nucleic acid sequence selected from SEQ ID NO: 1-23. According to another embodiment, the shRNA comprises a complementary strand comprising a nucleic acid sequence selected from SEQ ID NO: 24-46. According to some embodiments, the shRNA inhibiting the expression of PTEN comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii); SEQ ID NO: 22 and 45; or (xxiii) SEQ ID NO: 23 and 46. According to some embodiments, the shRNA comprises a guide strand and a sense strand comprising or consisting of the nucleic acid sequences SEQ ID NO: 2 and SEQ ID NO: 25, respectively. According to some embodiments, the shRNA comprises a guide strand and a sense strand comprising or consisting of the nucleic acid sequences SEQ ID NO: 3 and SEQ ID NO: 26, respectively. According to some embodiments, the shRNA comprises a guide strand and a sense strand comprising or consisting of the nucleic acid sequences SEQ ID NO: 4 and SEQ ID NO: 27, respectively. According to some embodiments, the shRNA is conjugated with cholesterol. According to some embodiments, the EVs are exosomes, microvesicles or a combination thereof. According to some embodiments, the EVs are derived from mesenchymal stem cells. According to some embodiments, the EVs are derived from bone marrow mesenchymal stem cells.

The siRNA and shRNA molecules promote sequence-specific degradation of mRNA to achieve inhibition of the expression of the desired protein gene, e.g., PTEN, or reduction of the expression level of the PTEN gene, e.g., by 95%, 90%, 85%, 80%, 75%, 70%, 60%, 50%, 40%, 30%, 20%, 10%, or 5%.

According to some embodiments, the EVs of the present invention comprise chondroitinase ABC (chABC enzyme) or a nucleic acid encoding the same.

A mesenchymal stem cell EVs may be produced by culturing mesenchymal stem cells in a medium to condition it. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or -mercaptoethanol, or any combination thereof.

The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrane. The conditioned medium may be concentrated about 50 times or more.

It will be appreciated that polynucleotides or oligonucleotides such as siRNA or shRNA may also be loaded directly into the EVs. In one embodiment, direct loading of RNAi oligonucleotide to the EVs is carried out by electroporation and/or with the use of transfection agents. In alternative embodiments, the loading is carried out in the absence of electroporation and/or in the absence of transfection agents.

According to one embodiment, the EVs are incubated with the RNAi oligonucleotide inhibitor for a period of time sufficient to permit loading of the particles with the nucleic acid based inhibitor. The duration of time sufficient to permit loading of the EVs with the nucleic acid based inhibitor cargo can be optimized for the particular type of cargo and if modified to comprise a hydrophobic modification, then the type of modification. Generally, an incubation of about 1 hour or less is sufficient to permit efficient loading of particles with nucleic acid cargo. In many instances, hydrophobically modified cargo is efficiently loaded into exosomes in a very rapid period of time, for example, within 5 minutes. Accordingly, in some embodiments, efficient loading takes place during an incubation period of 5 minutes or less, e.g., from 1-5 minutes. In exemplary embodiments, efficient loading takes place during an incubation period of 5 minutes, 10 minutes, 15 minutes, 20 minutes, 30 minutes, etc. In other embodiments, efficient loading may take place within 1 hour, within 2 hours, within 3 hours, within 4 hours, within 5 hours, within 6 hours, within 7 hours, within 8 hours, within 9 hours, within 10 hours, within 12 hours, within 24 hours, etc.

Loading of EVs with oligonucleotides is not highly temperature-dependent. In exemplary embodiments, exosomes are loaded at or around 37° C. In other embodiments, EVs (e.g., exosomes) can be loaded at or around room temperature. In other embodiments, exosomes can be loaded at or around 4° C.

According to some embodiments, the EVs can be loaded without the use of ultracentrifugation. According to other embodiment, the loading further comprises ultracentrifugation. According to some embodiment, the method of preparation further comprises a step of purification or isolation of the loaded EVs. According to one embodiment, the isolation is effected by centrifugation, e.g., ultracentrifugation. According to another embodiment, the isolation is effected via filtration. According to one embodiment, the ratio of EVs number to residual parent cells number following purification is at least 2, 3, 4, 5, 6, 8 or 10 times higher, or in certain advantageous embodiments at least 50, 100 or, 1000 times higher than in the initial material. According to some embodiment, the EVs are cell-free EVs.

According to some embodiments, the present invention provides a method of preparation of EVs, e.g., exosomes, the method comprises incubating EVs with conjugated RNAi oligonucleotides such as siRNA or shRNA for 0.5 to 5 hours at a temperature of 25 to 42° C. According to some embodiments, the conjugates of the siRNA or shRNA are conjugates with cholesterol.

According to one embodiment, the method further comprises a step of isolation of the loaded EVs using centrifugation, e.g., ultracentrifugation. According to some embodiments, another hydrophobic moiety may be used instead of cholesterol. According to one embodiment, the RNAi oligonucleotide is siRNA.

According to other embodiment, the EVs loaded with RNAi interference oligonucleotides as described herein may be obtained from cells artificially loaded with an RNAi oligonucleotide or with a polynucleotide encoding and capable of expressing or generating said RNAi interference oligonucleotides within a cell. In this case, the polynucleotide/oligonucleotide agent is ligated in a nucleic acid construct under the control of a cis-acting regulatory element (e.g., promoter) capable of directing an expression of the agent in a constitutive or inducible manner.

The nucleic acid agent may be delivered using an appropriate gene delivery vehicle/method (transfection, transduction, etc.). Optionally an appropriate expression system is used. Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co.

The expression construct may also be a virus. Examples of viral constructs include but are not limited to adenoviral vectors, retroviral vectors, vaccinia viral vectors, adeno-associated viral vectors, polyoma viral vectors, alphaviral vectors, rhabdoviral vectors, lentiviral vectors and herpesviral vectors.

A viral construct such as a retroviral construct includes at least one transcriptional promoter/enhancer or locus-defining element(s), or other elements that control gene expression by other means such as alternate splicing, nuclear RNA export, or post-transcriptional modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for the secretion of the peptide from the host cell in which it is placed. Preferably, the signal sequence for this purpose is a mammalian signal sequence or the signal sequence of the peptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof.

Preferably the viral dose for infection is at least 10³, 10⁴, 10⁵, 10⁶, 10⁷, 10⁸, 10⁹, 10¹⁰, 10¹¹, 10¹², 10¹³, 10¹⁴, 10¹⁵ or higher pfu or viral particles.

Double-stranded RNA may be synthesized by adding two opposing promoters to the ends of the gene segments, wherein one promoter is placed immediately 5′ to the gene and the opposing promoter is placed immediately 3′ to the gene segment. The dsRNA may then be transcribed with the appropriate polymerase.

In another embodiment, polynucleotide or oligonucleotide agents can be incubated with cells in culture, resulting in efficient uptake of the nucleic acid by cells. For such an embodiment, preferably the nucleic acid agents are hydrophobically modified, as further described herein below.

Irrespective of the method used to load the particles with the nucleic acid agents described herein, the cells are then incubated for a period of time sufficient for EVs, e.g., exosome, production. Exosomes isolated from the culture media contain exosomes loaded with the nucleic acid molecule taken up, produced or expressed by the cells. Accordingly, in one embodiment, a method of loading EVs with oligonucleotide cargo is provided, comprising incubating cells capable of EVs production (e.g., exosome production) with an oligonucleotide for a period of time sufficient for the oligonucleotide to be internalized by the cells, culturing the cells for a period of time sufficient for exosome secretion, and isolating exosomes loaded with the oligonucleotide from the culture medium.

According to some embodiments, the present invention provides isolated EVs prepared according to any one of the above embodiments.

According to yet another aspect, the present invention provides liposomes comprising the RNAi oligonucleotides as defined in any one of the above aspects and embodiments. All terms, embodiments and definitions disclosed in any one of the above aspects apply and are encompassed herein as well. According to some embodiments, the RNAi oligonucleotide inhibiting the expression of PTEN is shRNA or siRNA. According to some embodiments, the present invention provides liposomes comprising siRNA, shRNA or both inhibiting the expression of PTEN protein and comprising a guide strand comprising or consisting of a nucleic acid sequence selected from SEQ ID NO: 1-23. According to another embodiment, the siRNA and/or shRNA comprise a complementary strand comprising a nucleic acid sequence selected from SEQ ID NO: 24-46. According to some embodiments, the siRNA and/or shRNA inhibiting the expression of PTEN comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii); SEQ ID NO: 22 and 45; or (xxiii) SEQ ID NO: 23 and 46. According to some embodiments, the siRNA and/or shRNA comprises a guide strand and a sense strand comprising or consisting of the nucleic acid sequences SEQ ID NO: 2 and SEQ ID NO: 25, respectively. According to some embodiments, the siRNA and/or shRNA comprises a guide strand and a sense strand comprising or consisting of the nucleic acid sequences SEQ ID NO: 3 and SEQ ID NO: 26, respectively. According to some embodiments, the siRNA and/or shRNA comprises a guide strand and a sense strand comprising or consisting of the nucleic acid sequences SEQ ID NO: 4 and SEQ ID NO: 27, respectively. According to some embodiments, the shRNA is conjugated with cholesterol.

The term “liposome” is used herein as well-known in the art and refers to a microscopic closed vesicle having an internal phase enclosed by a lipid bilayer. A liposome can be a small single-membrane liposome such as a small unilamellar vesicle (SUV), large single-membrane liposome such as a large unilamellar vesicle (LUV), a still larger single-membrane liposome such as a giant unilamellar vesicle (GUV), a multilayer liposome having multiple concentric membranes such as a multilamellar vesicle (MLV), or a liposome having multiple membranes that are irregular and not concentric such as a multivesicular vesicle (MVV). The liposomes may be prepared as well known in the art.

According to another aspect, the present invention provides a pharmaceutical composition comprising a plurality of RNAi oligonucleotides as defined in any one of the above aspects and embodiments and/or a plurality of vesicles carrying the RNAi oligonucleotides, and a pharmaceutically acceptable carrier. According to some embodiments, the vesicles are EVs. According to other embodiments, the vesicles are liposomes. Non-limiting examples of vesicles are exosomes, liposomes, lipid nanoparticles, microvesicles, ectosomes, nanoparticles, nanocarriers, microparticles, or apoptotic bodies. Any one of the above definitions, terms and embodiments are encompassed and apply herein as well.

According to some embodiments, the present invention provides a pharmaceutical composition comprising a plurality of RNA interference (RNAi) oligonucleotide selected from siRNA and shRNA, comprising a guide strand comprising a nucleic acid sequence selected from SEQ ID NO: 1-23, for inhibiting the expression of phosphatase and tensin homolog (PTEN) protein, and a pharmaceutically acceptable carrier. According to some embodiments, the present invention provides a pharmaceutical composition comprising isolated EVs comprising RNA interference (RNAi) oligonucleotides inhibiting the expression of the protein PTEN, wherein the RNAi oligonucleotides comprise a guide strand comprising a nucleic acid sequence selected from SEQ ID NO: 1-23. According to some embodiments, the present invention provides a pharmaceutical composition comprising vesicles such as liposomes comprising RNAi oligonucleotides inhibiting the expression of the protein PTEN, wherein the RNAi oligonucleotides comprise a guide strand comprising a nucleic acid sequence selected from SEQ ID NO: 1-23. According to some embodiments, RNAi oligonucleotides are siRNA. According to some embodiments, RNAi oligonucleotides are shRNA. According to some embodiments, the siRNA or shRNA comprises a guide strand consisting of a nucleic acid sequence selected from SEQ ID NO: 1-23. According to some embodiments, the siRNA or shRNA comprises a sense (complementary) strand, i.e., a strand complementary to said guide strand. According to some embodiments, wherein the complementary strand is complementary to at least 14, 15, 16, 17, 18, 19, 20 or 21 contiguous nucleotides of the guide strand. As used herein, the term “complementary” refers to the ability of a first polynucleotide to hybridize to a second polynucleotide under certain conditions, such as stringent conditions. According to some embodiments, the complementary strand is complementary to from 14 to 19 contiguous nucleotides of the guide strand. According to some embodiments, the complementary strand comprises from 14 to 19 nucleotides. According to some embodiments, the complementary strand comprises from 14, 15, 16, 17, 18 or 19 nucleotides. According to some embodiments, the complementary strand comprises a nucleic acid sequence selected from SEQ ID NO: 24-46. According to some embodiments, the siRNA or shRNA comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii); SEQ ID NO: 22 and 45; or (xxiii) SEQ ID NO: 23 and 46. According to some embodiments, the siRNA or shRNA comprises a guide strand and a sense strand comprising or consisting of the nucleic acid sequences SEQ ID NO: 2 and SEQ ID NO: 25, respectively. According to some embodiments, the siRNA or shRNA comprises a guide strand and a sense strand comprising or consisting of the nucleic acid sequences SEQ ID NO: 3 and SEQ ID NO: 26, respectively. According to some embodiments, the siRNA or shRNA comprises a guide strand and a sense strand comprising or consisting of the nucleic acid sequences SEQ ID NO: 4 and SEQ ID NO: 27, respectively. According to one embodiment, siRNA or siRNA is conjugated with a hydrophobic moiety. According to some embodiments, the hydrophobic moiety is selected from the group consisting of a sterol, a ganglioside, a lipid, a vitamin, a fatty acid, a peptide, and a combination thereof. According to one embodiment, the siRNA or shRNA is conjugated with a sterol. In exemplary embodiments, the moiety is a sterol cholesterol molecule, therefore according to such embodiments, the siRNA or shRNA is conjugated with a cholesterol.

According to some embodiments, the present invention provides a pharmaceutical composition comprising EVs comprising siRNA molecules inhibiting the expression of PTEN comprising a guide strand comprising or consisting of a nucleic acid sequence selected from SEQ ID NO: 1-23. According to another embodiment, the siRNA comprises a complementary strand comprising a nucleic acid sequence selected from SEQ ID NO: 24-46. According to some embodiments, the siRNA comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (Xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii); SEQ ID NO: 22 and 45; or (xxiii) SEQ ID NO: 23 and 46. According to some embodiments, the present invention provides a pharmaceutical composition comprising EVs comprising siRNA molecules comprising a guide strand and a sense strand comprising or consisting of the nucleic acid sequences SEQ ID NO: 2 and SEQ ID NO: 25, respectively. According to some embodiments, the present invention provides a pharmaceutical composition comprising EVs comprising siRNA molecules comprising a guide strand and a sense strand comprising or consisting of the nucleic acid sequences SEQ ID NO: 3 and SEQ ID NO: 26, respectively. According to some embodiments, the present invention provides a pharmaceutical composition comprising EVs comprising siRNA molecules comprising a guide strand and a sense strand comprising or consisting of the nucleic acid sequences SEQ ID NO: 4 and SEQ ID NO: 27, respectively. According to some embodiments, the siRNA is conjugated with cholesterol. According to some embodiments, the EVs are exosomes, microvesicles or a combination thereof. According to some embodiments, the EVs are derived from mesenchymal stem cells.

According to some embodiments, the present invention provides a pharmaceutical composition comprising EVs comprising shRNA inhibiting the expression of PTEN comprising a guide strand comprising or consisting of a nucleic acid sequence selected from SEQ ID NO: 1-23. According to another embodiment, the shRNA comprises a complementary strand comprising a nucleic acid sequence selected from SEQ ID NO: 24-46. According to some embodiments, the shRNA comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii); SEQ ID NO: 22 and 45; or (xxiii) SEQ ID NO: 23 and 46. According to some embodiments, the shRNA is conjugated with cholesterol. According to some embodiments, the EVs are exosomes, microvesicles or a combination thereof. According to some embodiments, the EVs are derived from mesenchymal stem cells.

According to any one of the above embodiments, the shRNA or siRNA is loaded onto the vesicles, such as EVs.

The siRNA and shRNA molecules promote sequence-specific degradation of mRNA to achieve inhibition of the expression of the desired protein gene, or reduction of the expression level of the PTEN gene, e.g., by 95%, 90%, 85%, 80%, 75%, 70%, 60%, 50%, 40%, 30%, 20%, 10%, or 5%.

The term “pharmaceutical composition” as used herein refers to a composition comprising the active ingredient, i.e., RNAi oligonucleotide as described herein, either as is or loaded onto vesicles, in particular onto EVs such as exosomes, formulated together with one or more pharmaceutically acceptable carriers.

Formulation of the pharmaceutical composition may be adjusted according to applications. In particular, the pharmaceutical composition may be formulated using a method known in the art so as to provide rapid, continuous or delayed release of the active ingredient after administration to mammals. For example, the formulation may be any one selected from among plasters, granules, lotions, liniments, lemonades, aromatic waters, powders, syrups, ophthalmic ointments, liquids and solutions, aerosols, sprays, extracts, elixirs, ointments, fluidextracts, emulsions, suspensions, decoctions, infusions, ophthalmic solutions, tablets, suppositories, injections, spirits, capsules, creams, troches, tinctures, pastes, pills, and soft or hard gelatin capsules.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. solid carriers or excipients such as, for example, lactose, starch or talcum or liquid carriers such as, for example, water, fatty oils or liquid paraffin. Other examples of the carrier include culture media such as DMEM or RPMI; hypothermic storage medium containing components that scavenge free radicals, provide pH buffering, oncotic/osmotic support, energy substrates and ionic concentrations that balance the intracellular state at low temperatures; and mixtures of organic solvents with water.

According to any one of the above embodiments, the pharmaceutical composition is formulated for administration via an administration route selected from intranasal, intra-lesion, intrathecal, intravenous, intramuscular, subcutaneous, sublingual, oral, and intracerebral administration routes. According to one embodiment, the pharmaceutical composition is formulated for intranasal administration. According to some embodiment, such pharmaceutical composition is in the form of liquid solution, nasal drops, spray, and measured stray. According to other embodiments, the pharmaceutical composition is formulated for injection, e.g., intra-lesion, intrathecal or intravenous injection. According to such embodiments, the pharmaceutical composition is in the form of a sterile solution of injection.

According to some embodiments, the pharmaceutical composition is formulated for administration via an administration route selected from intranasal, intra-lesion, intrathecal, intravenous, intramuscular, subcutaneous, sublingual, oral, and intracerebral administration routes.

According to one embodiment, the pharmaceutical composition is formulated for intranasal administration.

According to some embodiments, the present invention provides an intranasal pharmaceutical composition comprising siRNA or shRNA molecules inhibiting the expression of PTEN protein comprising a guide strand comprising or consisting of a nucleic acid sequence selected from SEQ ID NO: 1-23. According to another embodiment, the siRNA or shRNA inhibiting the expression of PTEN protein comprises a complementary strand comprising a nucleic acid sequence selected from SEQ ID NO: 24-46. According to some embodiments, the siRNA or shRNA inhibiting the expression of PTEN comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii); SEQ ID NO: 22 and 45; or (xxiii) SEQ ID NO: 23 and 46. According to some embodiments, the siRNA or shRNA comprises a guide strand and a sense strand comprising or consisting of the nucleic acid sequences SEQ ID NO: 2 and SEQ ID NO: 25, respectively. According to some embodiments, the siRNA or shRNA comprises a guide strand and a sense strand comprising or consisting of the nucleic acid sequences SEQ ID NO: 3 and SEQ ID NO: 26, respectively. According to some embodiments, the siRNA or shRNA comprises a guide strand and a sense strand comprising or consisting of the nucleic acid sequences SEQ ID NO: 4 and SEQ ID NO: 27, respectively.

According to some embodiments, the present invention provides an intranasal pharmaceutical composition comprising EVs loaded with siRNA or shRNA molecules inhibiting the expression of PTEN protein comprising a guide strand comprising or consisting of a nucleic acid sequence selected from SEQ ID NO: 1-23. According to another embodiment, the siRNA or shRNA inhibiting the expression of PTEN protein comprises a complementary strand comprising a nucleic acid sequence selected from SEQ ID NO: 24-46. According to some embodiments, the siRNA or shRNA inhibiting the expression of PTEN comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii); SEQ ID NO: 22 and 45; or (xxiii) SEQ ID NO: 23 and 46. According to some embodiments, the siRNA or shRNA comprises a guide strand and a sense strand comprising or consisting of the nucleic acid sequences SEQ ID NO: 2 and SEQ ID NO: 25, respectively. According to some embodiments, the siRNA or shRNA comprises a guide strand and a sense strand comprising or consisting of the nucleic acid sequences SEQ ID NO: 3 and SEQ ID NO: 26, respectively. According to some embodiments, the siRNA or shRNA comprises a guide strand and a sense strand comprising or consisting of the nucleic acid sequences SEQ ID NO: 4 and SEQ ID NO: 27, respectively. According to some embodiments, the siRNA or shRNA is conjugated with cholesterol. According to some embodiments, the EVs are exosomes, microvesicles or a combination thereof. According to some embodiments, the EVs are derived from mesenchymal stem cells. According to some embodiments, the EVs are derived from bone marrow mesenchymal stem cells.

According to some embodiments, the pharmaceutical composition is formulated for local administration. Non-limiting examples of such formulations are ophthalmic formulations and formulations for injection.

According to some embodiments, the pharmaceutical composition of the present invention further comprises chondroitinase ABC (chABC enzyme).

According to any one of the above embodiments, the pharmaceutical composition of the present invention is useful for treating diseases, disorders or conditions requiring regeneration of cells. Thus, according to some embodiments, the pharmaceutical composition of the present invention is for use in regenerative therapy. According to some embodiments, the pharmaceutical composition according to any one of the above embodiments, is for use in treating neuroregeneration. According to some embodiments, the pharmaceutical composition according to any one of the above embodiments, is for use in treating a disease or condition selected from a neurodegenerative disease, neuronal disorder, neuronal injury or CNS damage. According to some embodiments, the pharmaceutical composition is for use in treating a neuronal injury or damage in a subject. According to some embodiments, the neuronal injury or damage is a spinal cord injury (SCI). According to some embodiments, the injury or damage are traumatic injury or damage. According to some embodiments, the pharmaceutical composition comprising the siRNA or shRNA comprising a guide strand and a sense strand comprising or consisting of the nucleic acid sequences SEQ ID NO: 2 and SEQ ID NO: 25, respectively, is for use in treating diseases, disorders or conditions requiring regeneration of cells as described above such as for use in treating a neuronal injury or damage in a subject. According to some embodiments, the pharmaceutical composition comprising the siRNA or shRNA comprising a guide strand and a sense strand comprising or consisting of the nucleic acid sequences SEQ ID NO: 3 and SEQ ID NO: 26, respectively, is for use in treating diseases, disorders or conditions requiring regeneration of cells as described above such as for use in treating a neuronal injury or damage in a subject. According to some embodiments, the pharmaceutical composition comprising the siRNA or shRNA comprising a guide strand and a sense strand comprising or consisting of the nucleic acid sequences SEQ ID NO: 4 and SEQ ID NO: 27, respectively, is for use in treating diseases, disorders or conditions requiring regeneration of cells as described above such as for use in treating a neuronal injury or damage in a subject.

The term “neurological disease, disorder or condition” refers to a disease, disorder or condition of the brain, spine and/or the nerves that connect them.

According to a particular embodiment, the condition is due to an injury. According to one embodiment, the injury is to the spinal cord, i.e., spinal cord injury (SCI). According to other embodiment, the neurological disease, disorder or condition is a neuronal damage or neuronal injury. According to some embodiments, the disease or damage is to the central neural system (CNS).

The terms “spinal cord injury” and “SCI” are used herein interchangeably and refer to an injury to the spinal cord. According to one embodiment, the injury is a result of a trauma. According to another embodiment, the injury or a damage is a result of a degeneration or a disease. Depending on where the spinal cord and nerve roots are damaged, the symptoms can vary widely, for example from pain to paralysis to incontinence. Spinal cord injuries are described at various levels of “incomplete”, which can vary from having no effect on the patient to a “complete” injury which means a total loss of function. Spinal cord injuries have many causes, but are typically associated with major trauma from motor vehicle accidents, falls, sports injuries, and violence. Thus, according to one embodiment, the SCI is selected from a complete and incomplete SCI. According to some embodiment, the spinal cord injury is selected from an acute or chronic SCI. The spinal cord injury may be susceptible to secondary tissue injury, including but not limited to: glial scarring, myelin inhibition, demyelination, cell death, lack of neurotrophic support, ischemia, free-radical formation, and excitotoxicity.

Diseases of the spinal cord include but are not limited to autoimmune diseases (e.g., multiple sclerosis), inflammatory diseases (e.g., Arachnoiditis), neurodegenerative diseases, polio, spina bifida and spinal tumors.

Subjects that may be treated according to the teaching of the present invention include mammalian subjects, such as humans, mice, rats, monkeys, dogs and cats. In one embodiment, the subject is a human subject.

The term “neurodegenerative disease or disorder” refers to any disease or disorder characterized by the dysfunction and/or death of neurons leading to a loss of neurologic function in the brain, spinal cord, central nervous system, and/or peripheral nervous system. Neurodegenerative diseases can be chronic or acute. Examples of neurodegenerative diseases include, but are not limited to, Alzheimer's disease, Parkinson's disease, frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, Pick's disease, Pick's disease-like dementia, Mild Cognitive Impairment, Diffuse Lewy body disease, Dementia with Lewy bodies type, demyelinating diseases such as multiple sclerosis and acute transverse myelitis, Balo's Concentric Sclerosis, Acute Disseminating Encephalomyelitis, Neuromyelitis Optica, Transverse Myelitis or Leukodystrophies, amyotrophic lateral sclerosis, Huntington's disease, Creutzfeldt-Jakob disease, AIDs dementia complex, extrapyramidal and cerebellar disorders such as lesions of the corticospinal system, disorders of the basal ganglia, corticobasal ganglionic degeneration, progressive supranuclear Palsy, structural lesions of the cerebellum, spinocerebellar degenerations, such as spinal ataxia, Friedreich's ataxia, cerebellar cortical degenerations, multiple systems degenerations (Mencel, Dejerine-Thomas, Shi-Drager, and Machado-Joseph), multiple system atrophy, systemic disorders (Refsum's disease, abetalipoprotemia, ataxia, telangiectasia, and mitochondrial multisystem disorder), disorders of the motor unit such as neurogenic muscular atrophies (anterior horn cell degeneration, infantile spinal muscular atrophy, and juvenile spinal muscular atrophy), Progressive Bulbar Palsy, Down's Syndrome in middle age, subacute sclerosing panencephalitis, Hallervorden-Spatz disease, dementia pugilistica, Primary Lateral Sclerosis, Progressive Pseudobulbar Palsy or Post-polio Syndrome; peripheral neuropathy is inherited (HNPP, CMTIA, CMT1B, DSS, CMT1X, CMT4B1), infectious (Leprosy, HIV), immune (GBS), diabetic (Type I, Type II), injury (transient nerve crush, chronic constriction injury, partial nerve ligation, spinal nerve ligation, spared nerve injury), and chemotherapy (e.g. cisplatin)-induced neuropathies; and the like.

The term “treating” a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting the development of symptoms characteristic of the disorder(s) being treated; (c) limiting worsening of symptoms characteristic of the disorder(s) being treated; (d) limiting recurrence of the disorder(s) in patients that have previously had the disorder(s); and/or (c) limiting recurrence of symptoms in patients that were previously asymptomatic for the disorder(s). According to some embodiments, the term “treating” comprises neural regeneration, axonal propagation, decreased astrogliosis and microgliosis at the injury site. According to other embodiments, the term encompasses improvement in symptoms associated with the disease or condition. According to one embodiment, the term “treating” comprises an improvement in locomotor parameters. According to one embodiment, improvement in locomotor parameters comprises improvement in 20%, 30%, 40%, 50%, 60%, 70%, 80% or 90% of locomotor parameters in comparison to untreated subjects. According to some embodiment, treating comprises reducing astrogliosis and/or microgliosis at the injury site. According to one embodiment, reducing astrogliosis and/or microgliosis comprises the reduction of 10%, 20%, 30%, 40%, 50%, 60%, 70%, or 80% of astrogliosis and/or microgliosis in comparison to untreated subject.

The pharmaceutical composition of the present invention may be administered using any known method. The terms “administering” or “administration of” a substance, a compound or an agent to a subject can be carried out using one of a variety of methods known to those skilled in the art. For example, a compound or an agent can be administered intranasally (e.g., by inhalation), intrathecally (into the spinal canal, or into the subarachnoid space), arterially, intradermally, intramuscularly, intraperitoneally, intravenously, subcutaneously, ocularly, sublingually, orally (by ingestion), intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. According to some embodiments, the composition is administered 1, 2, 3, 4, 5 or 6 times a day. According to other embodiments, the composition is administered 1, 2, 3, 4, 5 or 6 times a month. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. According to one embodiment, the pharmaceutical composition of the present invention is administered intranasally. According to another embodiment, the pharmaceutical composition of the present invention is administered intra-lesion. According to another embodiment, the pharmaceutical composition of the present invention is administered in proximity to the damage or injury. According to one embodiment, the pharmaceutical composition is administered orally. According to one embodiment, the pharmaceutical composition is administered intranasally. According to some embodiments, the pharmaceutical composition is administered locally. According to some embodiments, the pharmaceutical composition is administered systemically.

An exemplary dose of membrane vesicles (e.g., exosomes) that may be administered (e.g., intranasally) per treatment may be between 1×10⁶-1×10²⁰ and or between 1×10⁹-1×10¹⁵ for a 70 kg human.

According to another aspect, the present invention provides a method of treating a disease or condition associated with cell degeneration or cell death, comprising administering to the subject a therapeutically effective amount of RNAi oligonucleotides as described in any one of the above embodiments, extracellular vesicles comprising thereof or the pharmaceutical composition comprising same. According to some embodiments, a method comprises treating a neuronal injury or damage in a subject in need thereof. According to some embodiments, the method comprises administering to the subject a therapeutically effective amount of isolated EVs of the present invention comprising an RNA interference oligonucleotide of the present invention. According to some embodiments, wherein the administering is intranasal. According to other embodiments, the method further comprises administering chondroitinase ABC (chABC enzyme). The term “therapeutically effective amount” of the EVs, when administered to a subject will have the intended therapeutic effect, e.g., treating neuronal injury or damage such as SCI. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled person can readily determine the effective amount for a given situation by routine experimentation.

The term “therapeutically effective amount” of the membrane vesicles, when administered to a subject will have the intended therapeutic effect, e.g., treating neuronal injury such as SCI. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled person can readily determine the effective amount for a given situation by routine experimentation.

According to some embodiments, the membrane vesicles, such as EVs, are administered intranasal, intra-lesionally, parenterally, locally, systemically or orally.

According to another aspect, the present invention provides use of the RNAi oligonucleotides as described in any one of the above aspects and embodiments, for preparing a medicament for treating a disease or condition associated with cell degeneration or cell death.

In another aspect, the present disclosure provides a method for inhibiting or reducing the expression level of the PTEN gene and/or PTEN protein in a cell in vivo or in vitro, comprising introducing into the cell the siRNA or shRNA molecule of the invention as described herein above, the EVs or the pharmaceutical composition of the invention, such that the expression level of the PTEN gene is inhibited or reduced by at least 95%, at least 90%, at least 85%, at least 80%, at least 75%, at least 70%, at least 60%, at least 50%, at least 40%, at least 30%, at least 20%, at least 10%, or at least 5%. According to some embodiments, the siRNA or shRNA inhibiting the expression of PTEN molecules comprise a guide strand comprising or consisting of a nucleic acid sequence selected from SEQ ID NO: 1-23. According to another embodiment, the siRNA or shRNA comprises a complementary strand comprising a nucleic acid sequence selected from SEQ ID NO: 24-46. According to some embodiments, the siRNA or shRNA inhibiting the expression of PTEN comprises of a pair of oligonucleotides comprising or consisting of nucleic acid sequences (i) SEQ ID NO: 1 and 24; (ii) SEQ ID NO: 2 and 25; (iii) SEQ ID NO: 3 and 26; (iv) SEQ ID NO: 4 and 27; (v) SEQ ID NO: 5 and 28; (v) SEQ ID NO: 6 and 29; (vii) SEQ ID NO: 7 and 30; (viii) SEQ ID NO: 8 and 31; (ix) SEQ ID NO: 9 and 32; (x) SEQ ID NO: 10 and 33; (xi) SEQ ID NO: 11 and 34; (xii) SEQ ID NO: 12 and 35; (xiii) SEQ ID NO: 13 and 36; (xiv) SEQ ID NO: 14 and 37; (xv) SEQ ID NO: 15 and 38; (xvi) SEQ ID NO: 16 and 39; (xvii) SEQ ID NO: 17 and 40; (xviii) SEQ ID NO: 18 and 41; (xix) SEQ ID NO: 19 and 42, (xx) SEQ ID NO: 20 and 43; (xxi) SEQ ID NO: 21 and 44; (xxii); SEQ ID NO: 22 and 45; or (xxiii) SEQ ID NO: 23 and 46. According to some embodiments, the siRNA or shRNA comprises a guide strand and a sense strand comprising or consisting of the nucleic acid sequences SEQ ID NO: 2 and SEQ ID NO: 25, respectively. According to some embodiments, the siRNA or shRNA comprises a guide strand and a sense strand comprising or consisting of the nucleic acid sequences SEQ ID NO: 3 and SEQ ID NO: 26, respectively. According to some embodiments, the siRNA or shRNA comprises a guide strand and a sense strand comprising or consisting of the nucleic acid sequences SEQ ID NO: 4 and SEQ ID NO: 27, respectively. According to some embodiments, the siRNA or shRNA is conjugated with cholesterol. According to some embodiments, the EVs are exosomes, microvesicles or a combination thereof. According to some embodiments, the EVs are derived from mesenchymal stem cells.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. The term “consisting essentially of” means that the composition or component may include additional ingredients, but only if the additional ingredients do not materially alter the basic and novel characteristics of the claimed compositions or methods.

As used herein, the term “about”, when referring to a measurable value such as an amount, a temporal duration, and the like, is meant to encompass variations of +/−10%, or +/−5%, +/−1%, or even +/−0.1% from the specified value.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature.

### Materials and Methods

### Exosome Purification Protocol

Human MSCs were purchased from Lonza (Basel, Switzerland). Cells were cultured and expanded. Cells were cultured with exosome-free platelets lysate (Rabin Medical Center, Israel), and 2-3 days later, the medium was collected. The exosomes were purified using a standard differential centrifugation protocol, which involved isolating the culture fluid and centrifuging for 10 min at 300 g. The supernatant was recovered and centrifuged for 10 min at 2,000 g and then re-centrifuged for 30 min at 10,000 g. The supernatant was then passed through a 0.22 μm filter, and centrifuged for 180 min at 100,000 g. The pellet, containing the exosomes and proteins, was washed three times using 30 kDa Amicon filters. Exosomes were characterized using NanoSight technology, electron microscopy and Western blotting for calnexin, as a negative marker, and CD9 and CD81, CD63, TSG101 as positive marker.

### Example 1

Several hundreds of RNA sequences, directed to binding to mRNA encoding PTEN protein, that were considered useful for siRNA preparation were ranked according to predetermined criteria. 23 of these sequences having the highest score were prepared and tested. Interestingly, the siRNA used in WO2019/186558 (antisense UUCUGUUUGUGGAAGAACUC (SEQ ID NO: 47 and sense GAGUUCUUCCACAAACAGAA SEQ ID NO: 48, also referred to as proof of concept (POC) sequence below) was ranked at 489 place. The sequences of the antisense (guide) and sense oligonucleotides of the siRNAs are provided in Table 1. In some cases, the sequence complementary to Guide polynucleotides comprises from 14 to 19 nucleotides

TABLE 1

| | Antisense | SEQ | |
|---|---|---|---|
| | (A)/ | ID | |
| SiRNA ID | Sense (S) | No: | Sequence (5′-3′) |
| sirna_1917 | A | 1 | AAGUUGAACUGCUAGCCUCUG |
| | S | 24 | GAGGCUAGCAGUUCAACUU |
| sirna_1455 | A | 2 | UUCCGCCACUGAACAUUGGAA |
| | S | 25 | CCAAUGUUCAGUGGCGGAA |
| sirna_1962 | A | 3 | AUCUAUAAUGAUCAGGUUCAU |
| | S | 26 | GAACCUGAUCAUUAUAGAU |
| sirna_1458 | A | 4 | AAGUUCCGCCACUGAACAUUG |
| | S | 27 | AUGUUCAGUGGCGGAACUU |
| sirna_1454 | A | 5 | UCCGCCACUGAACAUUGGAAU |
| | S | 28 | UCCAAUGUUCAGUGGCGGA |
| sirna_1459 | A | 6 | CAAGUUCCGCCACUGAACAUU |
| | S | 29 | UGUUCAGUGGCGGAACUUG |
| sirna_1457 | A | 7 | AGUUCCGCCACUGAACAUUGG |
| | S | 30 | AAUGUUCAGUGGCGGAACU |
| sirna_1456 | A | 8 | GUUCCGCCACUGAACAUUGGA |
| | S | 31 | CAAUGUUCAGUGGCGGAAC |
| sirna_1351 | A | 9 | ACAUAGCGCCUCUGACUGGGA |
| | S | 32 | CCAGUCAGAGGCGCUAUGU |
| sirna_1949 | A | 10 | AGGUUCAUUGUCACUAACAUC |
| | S | 33 | UGUUAGUGACAAUGAACCU |
| sirna_1466 | A | 11 | AGGAUUGCAAGUUCCGCCACU |
| | S | 34 | UGGCGGAACUUGCAAUCCU |
| sirna_1918 | A | 12 | GAAGUUGAACUGCUAGCCUCU |
| | S | 35 | AGGCUAGCAGUUCAACUUC |
| sirna_1950 | A | 13 | CAGGUUCAUUGUCACUAACAU |
| | S | 36 | GUUAGUGACAAUGAACCUG |
| sirna_1349 | A | 14 | AUAGCGCCUCUGACUGGGAAU |
| | S | 37 | UCCCAGUCAGAGGCGCUAU |
| sirna_1464 | A | 15 | GAUUGCAAGUUCCGCCACUGA |
| | S | 38 | AGUGGCGGAACUUGCAAUC |
| sirna_1370 | A | 16 | CAGGUAGCUAUAAUAAUACAC |
| | S | 39 | GUAUUAUUAUAGCUACCUG |
| sirna_1907 | A | 17 | GCUAGCCUCUGGAUUUGACGG |
| | S | 40 | GUCAAAUCCAGAGGCUAGC |
| sirna_1347 | A | 18 | AGCGCCUCUGACUGGGAAUAG |
| | S | 41 | AUUCCCAGUCAGAGGCGCU |
| sirna_1975 | A | 19 | GUGGUGUCAGAAUAUCUAUAA |
| | S | 42 | AUAGAUAUUCUGACACCAC |
| sirna_1677 | A | 20 | CUGGUCCUGGUAUGAAGAAUG |
| | S | 43 | UUCUUCAUACCAGGACCAG |
| sirna_1491 | A | 21 | CCUUUAGCUGGCAGACCACAA |
| | S | 44 | GUGGUCUGCCAGCUAAAGG |
| sirna_1453 | A | 22 | CCGCCACUGAACAUUGGAAUA |
| | S | 45 | UUCCAAUGUUCAGUGGCGG |
| sirna_1973 | A | 23 | GGUGUCAGAAUAUCUAUAAUG |
| | S | 46 | UUAUAGAUAUUCUGACACC |

In the sequence listing file, the letter “t” represents uracyl (U).

### Example 2

SH-SY5Y cells were cultured in 24-well TC plates until 70-80% confluency. Cells were transfected with each of the siRNA duplexes (at 50 pmol/well) using Lipofectamine™ RNAiMAX Transfection Reagent (Thermo Fisher) according to the manufacturer's protocol. After 36 hours, cells were collected and RNA was extracted using an RNeasy mini kit (QIAGEN) according to the manufacturer's protocol. cDNA was prepared from the abovementioned cells' lysate using High-Capacity cDNA Reverse Transcription Kit (Thermo Fisher). Realtime quantitative PCR was conducted on the QuantStudio 12K Flex real-time PCR system. Appropriate primers for PTEN (Thermo Fisher, assay ID: Hs02621230_s1) or GAPDH (Thermo Fisher, assay ID: Hs02786624_g1) were added. The amplification reaction conditions: 95° C. for 20 s, 40 cycles of 95° C. for 3 s, 60° C. for 30 s, in 10 μL reactions, in triplicates. The AACt method was used to determine relative expression levels, where the gene of interest was normalized to GAPDH expression.

Since the commercially available sequence that was considered as a reference (named here siRNA reference and used as our positive control) contained a cholesterol moiety that is aimed to enhance the efficacy of its transfection, each and every siRNA molecule was tested with and without conjugated cholesterol to determine if its role was significant for the transfection of our molecules.

The sequence named siRNA_pten5 is defined in Table 2.

TABLE 2
Pten5 (control) sequence

| | SED | Antisense | |
|---|---|---|---|
| siRNA duplex | ID | (A)/ | |
| ID | NO: | Sense (S) | Sequence (5′- 3′) |
| sirna_pten5/ | 49 | A | UUCUGUUUGUGGAAGAACUC |
| reference | 50 | S | CUUCCACAAACAGAA |

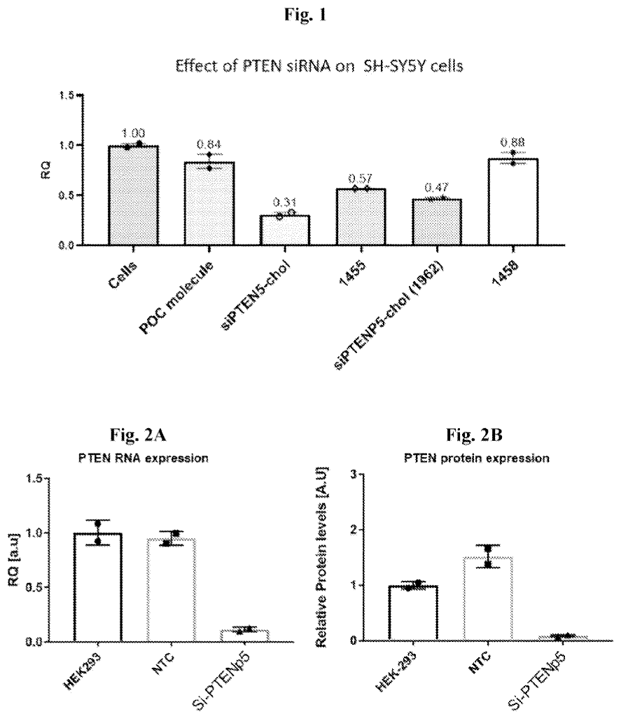

The results are presented in FIG. 1. The Relative Quantification (RQ) represented on the Y axis is equivalent to the 2^{-ΔΔCt} value. The RQ is the fold change compared to the calibrator (in this case-untreated cells). The calibrator has an RQ value of 1. All samples are compared to the calibrator. An RQ of 10 means that this gene is 10 times more expressed in sample x than in the calibrator sample. Pten5 (control), 1455, 1962, and 1458 are coded names for the four different PTEN siRNA molecules tested with and without cholesterol.

It can be seen from the Examples that the siRNA molecules tested have a higher ability of inhibition compared to the siRNA reference.

### Example 3. Effect of siRNA_1962 on PTEN Protein Expression in Human Embryonic Kidney Cells

The effect of siRNA_1962 on PTEN protein expression was in Human Embryonic Kidney cells (HEK293: a non-cancerous cell line).

HEK 293 cells were transfected with siRNA/NTC molecules using Lipofectamine 3000 (ThermoFisher) according to the manufacturer's protocol. The cells were then harvested in ice-cold PBS 96 Hours post-transfection.

RNA was extracted using RNeasy mini kit (QIAGEN) and cDNA was synthesized using High Capacity cDNA kit (Thermo). qPCR was performed using TaqMan probes (Thermo), for the determination of PTEN and GAPDH expression levels.

Protein was extracted with nonionic lysis detergent (NP-40 lysis buffer). Protein extracts were separated in SDS-PAGE gel electrophoresis under denatured conditions, then transferred to a nitrocellulose membrane with Trans-Blot Turbo Transfer System (BIO-RAD). The membrane was blocked with EveryBlot blocking buffer (BIO-RAD) and exposed to anti-PTEN mouse-monoclonal antibody (Santa Cruz) for ON at 4° C. In order to visualize the signal, the membrane was exposed to anti-mouse IgG HRP antibody for an hour, then provided with the substrate for enhanced chemiluminescence (ECL kit). Signal detection was done in LAS4000. The membrane was then washed and exposed to anti GAPDH rabbit-polyclonal antibody to determine the internal control for an hour at RT, then to an anti-rabbit IgG HRP. The signal was detected with LAS4000.

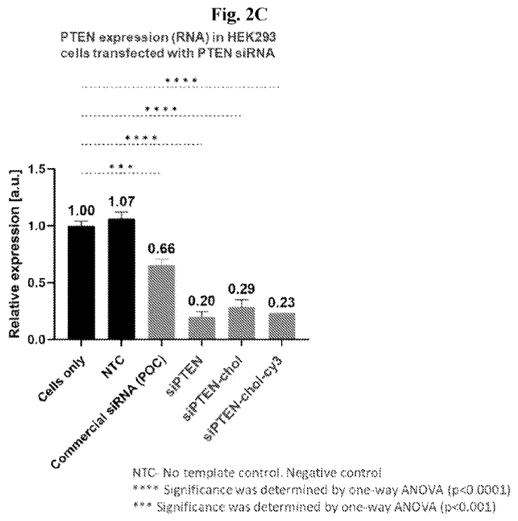

The results are presented in FIGS. 2A and 2B. It can be seen that siRNA_1962 decreases RNA and protein levels significantly; the level of PTEN RNA was reduced by ˜80% and PTEN protein levels by about 90%. Interestingly, the commercially available anti-PTEN siRNA, denoted herein as POC siRNA showed a much weaker effect FIGS. 2C and 2D. As seen in the figures, siRNA_1962 decreased PTEN RNA and protein levels significantly compared to POC siRNA. As shown in FIG. 2D, siRNA_1962 reduces the expression of PTEN RNA about 3 times more efficiently than the POC siRNA. The experiment was repeated at least 4 times. FIGS. 2E and 2F show the effect of siRNA_1962 on PTEN protein expression.

### Example 3

Further, the effect of siRNA_1962 (conjugated with cholesterol) loaded into extracellular vesicles (EVs) was tested for its efficacy to inhibit PTEN. The experiment was performed on HEK293 cells by incubating the EVs with cells

Loading of the exosomes includes incubation of the siRNA sequence (0.1 nmol) with 40 μl of BM-MSC derived exosomes (1*10E7 particles/μl) at 30° C. for 4 hours and washing the unloaded siRNA by 30kda amicon filter, or 100,000G ultracentrifugation for 2 hours.

### Example 4. In Vivo Efficacy of siRNA_1962

Rats were operated on for a complete transection of the spinal cord a T10 and divided into four treatments groups:
1. Exo—siRNA PTEN_cholesterol-exosomes loaded with siRNA_1962 siRNA conjugated with cholesterol (n=4)
2. siRNA_1962 siRNA conjugated with cholesterol (n=3)
3. Exosomes only (n=4)
4. Saline (n=6)

Rats received treatment for 5 days starting on the day of surgery, had 2 days break, and then received treatment for another 5 days.

From 1 week after the surgery, rats exercised on a treadmill 5 days a week and were tested for Dorsal Von Frey, weighed, and recorded walking for BBB scoring weekly. During the 10^{th} week, rats' spinal cords were scanned by MRI.

### Results

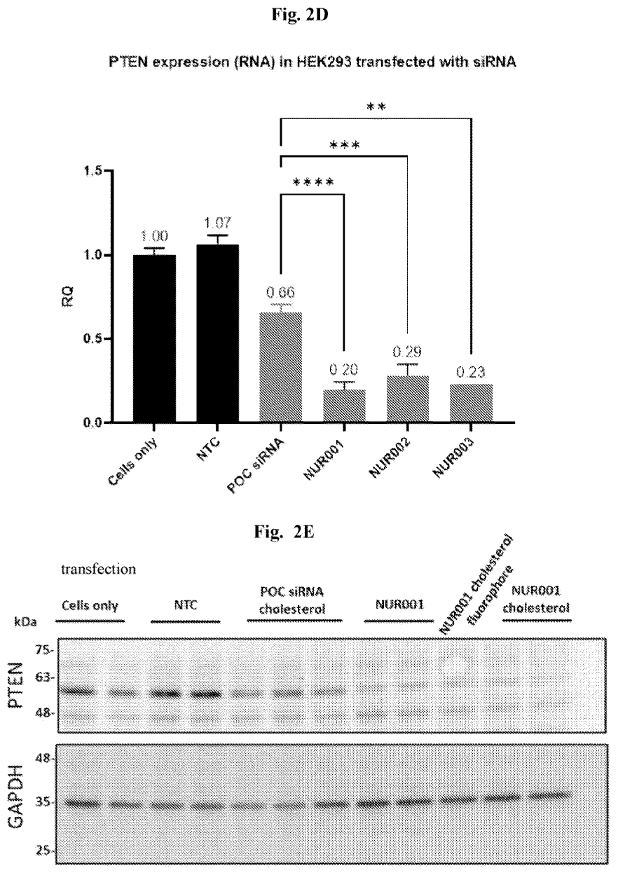

Sensory recovery was tested by Von Frey filament with a gradient of bending forces that were applied on the dorsal hindlimbs to determine the paw withdrawal threshold, as an indicator of sensory recovery. As can be seen in FIG. 3, one week after the surgery, the sensory response reached 75% in the treatment groups with Exo-PTEN_chol and 33% or less in the control groups. From the second week, the percentage of sensory recovered rat legs stayed the same as after 1 week until the end of the experiment. None of the rats in the saline group achieved any sensory recovery.

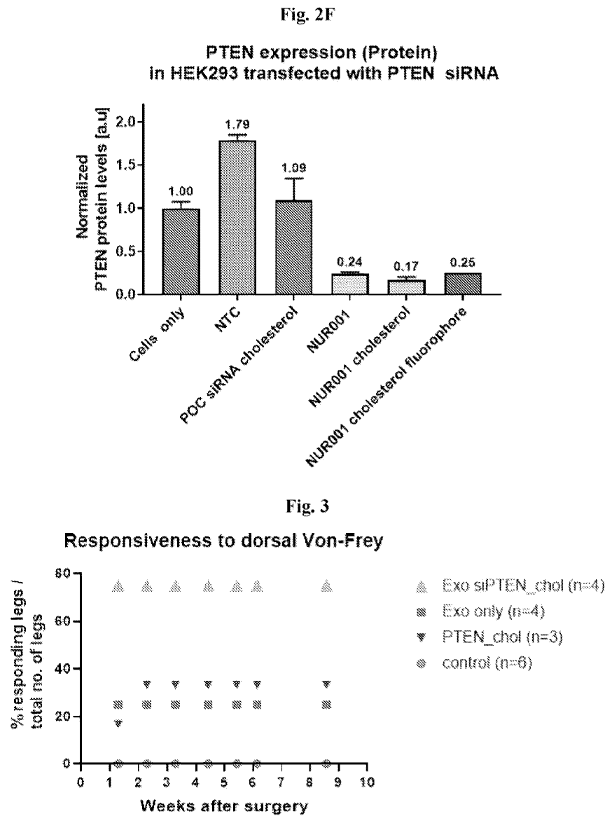

Reflexes recovery was tested by tail and paw pinch 2 weeks after surgery and the results are presented in. After complete transection SCI surgery, all rats lose the paw pinch reflex. We examined whether the treatment affects their reflex recovery, immediately after the rats completion of 2 weeks of treatment after the surgery. Our results show that the percentage of rats regaining paw pinch reflex 2 weeks after surgery is much higher in rats treated with Exo PTEN_chol (75%) compared to rats treated with Exo only (25%) PTEN_chol (33%), and control (0%) (see FIG. 4). Following the second week, the recovered reflex was unchanged in all the animals until the end of the experiment.

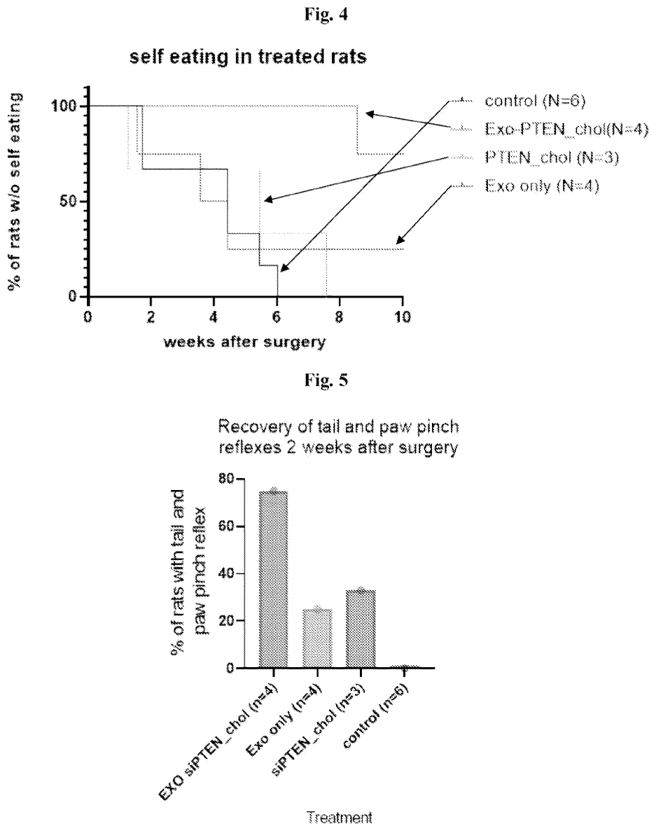

The well-being of rats was evaluated by the self-eating tendency of treated rats following the injury. Rats operated on for SCI are prone to self-eating. We have followed self-eating behavior in our rats and found (FIG. 5) that the percentage of rats without self-eating behavior in the Exo-PTEN_chol treatment group was significantly higher than in the control group (p-value=0.0033) and PTEN_chol group (p-value=0.01), but not significantly higher than in the Exo only group (p value=0.1).

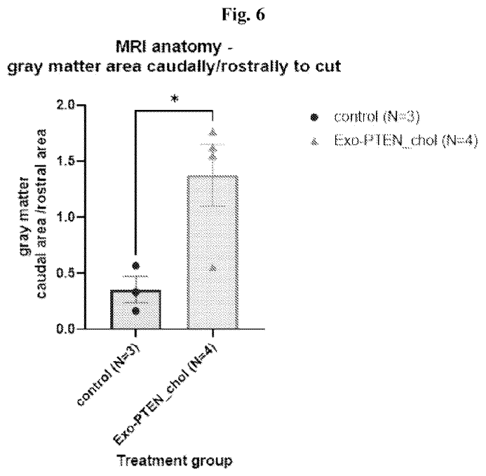

Sagittal MRI images, axial sections, and cross-sectional area images 4 mm caudal and rostral to the T10 epicenter in healthy rats or to the injury epicenter in untreated, exosome-treated, or ExoPTEN-treated rats were analyzed and the ratio of caudal to rostral area calculated, representing the regeneration of the tissue downstream the injury. ExoPTEN-treated (n=3), and healthy (n=3) rats. The results are shown in FIG. 6. In vivo MRI imaging clearly indicates neural tissue regeneration

Although the present invention has been described herein above by way of preferred embodiments thereof, it can be modified, without departing from the spirit and nature of the subject invention as defined in the appended claims.

1. An RNA interference (RNAi) oligonucleotide selected from siRNA and shRNA, comprising a guide strand comprising a nucleic acid sequence selected from SEQ ID NO: 1-23, wherein the RNAi oligonucleotide inhibits the expression of phosphatase and tensin homolog (PTEN) protein.

2. The RNAi oligonucleotide according to claim 1, wherein the RNAi oligonucleotide is siRNA and wherein the guide strand consists of a nucleic acid sequence selected from SEQ ID NO: 1-23.

3. The RNAi oligonucleotide according to claim 1, comprising a sense strand complementary to said guide strand, wherein the complementary strand is complementary to at least 14, 15, 16, 17, 18, 19, 20 or 21 contiguous nucleotides of the guide strand.

4. The RNAi oligonucleotide according to claim 3 wherein the sense strand consists of from 14 to 21 nucleotides.

5. The RNAi oligonucleotide according to claim 3, wherein the complementary strand comprises a nucleic acid sequence selected from SEQ ID NO: 24-46.

6. The RNAi oligonucleotide according to claim 1, wherein the RNAi oligonucleotide is siRNA comprising a guide strand comprising the nucleic acid sequence AUCUAUAAUGAUCAGGUUCAU (SEQ ID NO: 3) and a sense strand comprising the nucleic acid sequence GAACCUGAUCAUUAUAGAU (SEQ ID NO: 26).

7. The RNAi oligonucleotide according to claim 6, wherein the RNAi oligonucleotide is siRNA comprising a guide strand consisting of the nucleic acid sequence AUCUAUAAUGAUCAGGUUCAU (SEQ ID NO: 3) and a sense strand consisting of the nucleic acid sequence GAACCUGAUCAUUAUAGAU (SEQ ID NO: 26).

8. The RNAi oligonucleotide according to claim 1, conjugated with a loading moiety.

9. The RNAi oligonucleotide according to claim 8, wherein said loading moiety is selected from the group consisting of a sterol, a ganglioside, a lipid, a vitamin, a fatty acid, a hydrophobic peptide, and a combination thereof.

10. Isolated extracellular vesicles comprising RNAi oligonucleotides inhibiting the expression of PTEN protein according to claim 1.

11. The isolated extracellular vesicles according to claim 10, wherein the extracellular vesicles are loaded with the RNAi oligonucleotides ex vivo.

12. The isolated extracellular vesicles according to claim 10, wherein the extracellular vesicles are selected from exosomes, macrovesicles, microvesicles and a combination thereof.

13. The isolated extracellular vesicles according to claim 10, wherein said extracellular vesicles are derived from adherent cells expressing mesenchymal markers.

14. The isolated extracellular vesicles according to claim 13, wherein the adherent cells expressing mesenchymal markers are selected from mesenchymal stem cells and olfactory ensheathing cells.

15. A pharmaceutical composition comprising the RNAi oligonucleotides according to claim 1 or the extracellular vesicles comprising said RNAi oligonucleotide, and a pharmaceutically acceptable carrier and/or excipient.

16. The pharmaceutical composition according to claim 15, formulated for administration via an administration route selected from intranasal, intra-lesion, intrathecal, intravenous, intramuscular, subcutaneous, sublingual, oral, transdermal, topical, local, and intracerebral administration route.

17. (canceled)

18. (canceled)

19. (canceled)

20. (canceled)

21. (canceled)

22. (canceled)

23. (canceled)

24. A method of treating a disease or condition associated with cell degeneration or death, comprising administering to the subject a therapeutically effective amount of RNAi oligonucleotides according to claim 1, extracellular vesicles comprising said RNAi oligonucleotides or a pharmaceutical composition comprising said RNAi oligonucleotides or said extracellular vesicles.

25. The method according to claim 24, wherein the disease or condition is selected from a neurodegenerative disease, neuronal disorder, neuronal injury or CNS damage in a subject.

26. The method according to claim 25, wherein the neuronal injury or damage is a spinal cord injury (SCI).

27. The method according to claim 25, wherein the administering comprises intranasal or local administering.
